(19) United States
(12) Patent Application Publication
(10) Pub. No.: US 2022/0346993 A1
(43) Pub. Date: Nov. 3, 2022

(54) APPARATUS AND METHODS FOR LOADING AND DEPLOYING IMPLANTS FROM DELIVERY APPARATUSES

(71) Applicant: Edwards Lifesciences Corporation, Irvine, CA (US)

(72) Inventors: HARISH MANICKAM SRINIMUKESH, Costa Mesa, CA (US); Matthew Michael Becerra, Lake Forest, CA (US); Mark Chau, Laguna Hills, CA (US); Jesse Robert Edwards, Silverado, CA (US); Garrett Dallas Johnson, Costa Mesa, CA (US); David Robert Landon, Huntington Beach, CA (US); Ju Nie Leong, Garden Grove, CA (US); Hieu Minh Luong, Westminster, CA (US); Hiroshi Okabe, Costa Mesa, CA (US); Matthew A. Peterson, Costa Mesa, CA (US); Kevin M. Stewart, Powell, TN (US); Julio Cesar Sanchez, Garden Grove, CA (US)

(73) Assignee: Edwards Lifesciences Corporation, Irvine, CA (US)

(21) Appl. No.: 17/862,271

(22) Filed: Jul. 11, 2022

Related U.S. Application Data
(63) Continuation of application No. PCT/US2021/013123, filed on Jan. 12, 2021.
(60) Provisional application No. 62/966,891, filed on Jan. 28, 2020.

(51) Int. Cl. A61F 2/95

(52) U.S. Cl. CPC A61F 2/9525

(57) ABSTRACT

Apparatuses and methods for loading and deploying implants from delivery apparatuses. The apparatuses in certain embodiments may comprise crimping devices for compressing an implant for insertion into a portion of a delivery apparatus. The apparatuses in certain embodiments may comprise features of delivery apparatuses allowing for improved loading and deployment of an implant from the delivery apparatuses.

### CROSS REFERENCE TO RELATED APPLICATIONS

This application is a continuation of International Application No. PCT/US2021/013123, filed Jan. 12, 2021, which designates the United States and was published in English by the International Bureau on Aug. 5, 2021 as WO2021/154493, which claims priority to U.S. Provisional App. No. 62/966,891, filed Jan. 28, 2020, the entire contents of each of which are hereby incorporated by reference.

### BACKGROUND

### Field

Certain embodiments disclosed herein relate to apparatuses and methods for loading and deploying implants from delivery apparatuses. The apparatuses in certain embodiments may comprise crimping devices for compressing an implant for insertion into a portion of a delivery apparatus. The apparatuses in certain embodiments may comprise features of delivery apparatuses allowing for improved loading and deployment of an implant from the delivery apparatus, among other features.

### Background

Human heart valves, which include the aortic, pulmonary, mitral and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves' ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures. Such replacement valves often include a tissue-based valve body that is connected to an expandable frame that is then delivered to the native valve's annulus.

Development of prostheses including but not limited to replacement heart valves that can be compacted for delivery and then controllably expanded for controlled placement has proven to be particularly challenging. Delivering a prosthesis to a desired location in the human body, for example delivering a replacement heart valve to the mitral valve, can be challenging. A delivery apparatus may be provided to deploy such an implant to the desired location in the human body. The implant may be in a compressed state within the delivery apparatus, and thus must be compressed for insertion into the delivery apparatus. Further, release of the implant from the delivery apparatus may produce significant force that may impede the ability of the implant to be released from the delivery apparatus.

### SUMMARY

Embodiments of the present disclosure may include crimping devices for an implant. In certain embodiments, a crimping device may include a body having an inner surface forming a funnel extending from a wide end portion to a narrow end portion and surrounding a cavity for receiving the implant, the body including an opening at the narrow end portion and including a plurality of guide channels each configured for a portion of the implant to slide in to guide the implant at the opening.

A method may include sliding an implant within a cavity of a funnel in a direction from a wide end portion of the funnel to a narrow end portion of the funnel, an opening being positioned at the narrow end portion of the funnel. The method may include guiding an end portion of the implant at the opening by sliding at least a portion of the implant within guide channels coupled to the funnel.

Embodiments of the present disclosure may include crimping devices for an implant. In certain embodiments, a crimping device may include a coupler. The crimping device may include a plurality of elongate lever arms each having a distal end portion pivotally coupled to the coupler and a proximal end portion and configured to pivot about the coupler from an expanded state in which the plurality of elongate lever arms surround an implant receiving region to a reduced state in which the proximal end portions are moved closer to each other to compress the implant within the implant receiving region.

A method may include positioning an implant within an implant receiving region of a crimping device, the crimping device including a coupler and a plurality of elongate lever arms each having a distal end portion pivotally coupled to the coupler and a proximal end portion and configured to pivot about the coupler from an expanded state in which the plurality of elongate lever arms surround the implant receiving region to a reduced state in which the proximal end portions are moved closer to each other to compress the implant within the implant receiving region. A method may include compressing the implant within the implant receiving region by moving the plurality of elongate lever arms to the reduced state.

Embodiments of the present disclosure may include crimping devices for an implant. In certain embodiments, a crimping device may include at least one roller surrounding a channel configured for the implant to be passed through, the at least one roller configured to press against the implant to compress the implant and roll as the implant passes through the channel.

A method may include rolling at least one roller over at least a portion of an implant to compress the implant.

Embodiments of the present disclosure may include crimping devices for an implant. In certain embodiments, a crimping device may include a spiral body having an interior diameter and extending around an axis and surrounding a channel configured to receive the implant, the spiral body configured to rotate about the axis to reduce the interior diameter and compress the implant within the channel.

A method may include rotating a spiral body about an axis that the spiral body extends around to compress an implant with the spiral body, the implant positioned within a channel that the spiral body surrounds.

Embodiments of the present disclosure may include delivery systems for an implant. In certain embodiments, a delivery system may include an elongate shaft having a capsule surrounding an implant retention area for retaining the implant, the capsule having an opening for the implant to be deployed from. A delivery system may include a port configured to allow for fluid transfer into or out of the capsule.

A method may include inserting an implant into a capsule of a delivery apparatus through an opening of the capsule. The capsule may be configured to surround an implant retention area for retaining the implant. In certain embodiments, a method may include passing fluid out of the capsule through a port coupled to the delivery apparatus upon the implant being inserted into the capsule.

A method may include releasing an implant from a capsule of a delivery apparatus through an opening of the capsule. The capsule may be configured to surround an implant retention area for retaining the implant. A method may include passing fluid into the capsule through a port coupled to the delivery apparatus upon the implant being released from the capsule.

Embodiments of the present disclosure may include delivery systems for an implant. In certain embodiments, a delivery system may include an elongate shaft having a capsule surrounding an implant retention area for retaining the implant, the capsule including a plurality of layers each configured to be retracted separately to deploy the implant from the implant retention area.

A method may include separately retracting a plurality of layers of a multi-layer capsule extending over an implant retention area of a delivery apparatus to deploy the implant from the implant retention area.

Embodiments of the present disclosure may include delivery systems for an implant. In certain embodiments, a delivery system may include an elongate shaft having a capsule surrounding an implant retention area for retaining the implant, the capsule including an interior surface configured to face towards the implant within the implant retention area and including a plurality of protrusions for the implant to slide along.

A method may include sliding an implant relative to a capsule of a delivery apparatus, the capsule having an interior surface including a plurality of protrusions that the implant slides along.

Embodiments of the present disclosure may include delivery systems for an implant. In certain embodiments, a delivery system may include an elongate shaft having a capsule surrounding an implant retention area for retaining the implant, the capsule being configured to contour to a shape of the implant retained within the implant retention area.

Embodiments of the present disclosure may include delivery systems for an implant. In certain embodiments, a delivery system may include an elongate shaft having a capsule having a length and surrounding an implant retention area for retaining the implant, the capsule having an outer surface with a non-uniformly circular cross-sectional shape along the length of the capsule.

A method may include deploying an implant from an implant retention area surrounded by a capsule of a delivery apparatus, the capsule having a shape that contours to a shape of the implant within the implant retention area.

A method may include deploying an implant from an implant retention area surrounded by a capsule of a delivery apparatus, the capsule having an outer surface with a non-uniformly circular cross-sectional shape along a length of the capsule.

Embodiments of the present disclosure may include delivery systems for an implant. In certain embodiments, a delivery system may include an elongate shaft having an implant retention area for retaining the implant. The elongate shaft may include an elongate flat braid ribbon extending along the elongate shaft.

A method may include delivering an elongate shaft of a delivery system to an implantation site, the elongate shaft including an implant retention area for retaining an implant for deployment to the implantation site, and the elongate shaft including an elongate flat braid ribbon extending along the elongate shaft.

Embodiments of the present disclosure may include delivery systems for an implant. In certain embodiments, a delivery system may include an elongate shaft having an implant retention area for retaining the implant. The delivery system may include a hypotube coupled to the elongate shaft and including a first portion having a spiral cut pattern with a first pitch, a second portion having a spiral cut pattern with a second pitch that is different from the first pitch, and a third portion having a spiral cut pattern with a third pitch that is different from the first pitch and the second pitch.

A method may include delivering an elongate shaft of a delivery system to an implantation site, the elongate shaft including an implant retention area for retaining an implant for deployment to the implantation site, and the elongate shaft includes a hypotube coupled to the elongate shaft and including a first portion having a spiral cut pattern with a first pitch, a second portion having a spiral cut pattern with a second pitch that is different from the first pitch, and a third portion having a spiral cut pattern with a third pitch that is different from the first pitch and the second pitch.

### BRIEF DESCRIPTION OF THE DRAWINGS

Features and advantages of the systems, apparatuses, and methods as disclosed herein will become appreciated as the same become better understood with reference to the specification, claims, and appended drawings wherein:

FIG. 1 illustrates a cross sectional view of a crimping device according to an embodiment of the present disclosure.

FIG. 2 illustrates a top perspective view of an upper body of a crimping device shown in FIG. 1.

FIG. 3 illustrates a bottom view of the upper body shown in FIG. 2.

FIG. 4 illustrates a side perspective view of a side of the upper body shown in FIG. 2.

FIG. 5 illustrates a side perspective view of a pressing body shown in FIG. 1.

FIG. 6 illustrates a side perspective view of an actuator shown in FIG. 1.

FIG. 7 illustrates a cross sectional view along line 7-7 of the actuator in FIG. 6.

FIG. 8A illustrates a perspective view of an implant according to an embodiment of the present disclosure.

FIG. 8B illustrates a bottom view of the implant shown in FIG. 8A.

FIG. 9 illustrates a cross sectional view of the crimping device shown in FIG. 1 including an implant according to an embodiment of the present disclosure.

FIG. 10 illustrates a side perspective view of guide channels according to an embodiment of the present disclosure.

FIG. 11 illustrates a top view of the upper body shown in FIG. 2 with struts extending from a central opening.

FIG. 12 illustrates a side view of the crimping device shown in FIG. 1 over a portion of a delivery apparatus.

FIG. 13 illustrates a side perspective view of guide channels according to an embodiment of the present disclosure.

FIG. 14 illustrates a side perspective view of guide channels according to an embodiment of the present disclosure.

FIG. 15 illustrates a cross sectional view of a crimping device according to an embodiment of the present disclosure.

FIG. 16 illustrates a top perspective view of an upper body shown in FIG. 15.

FIG. 17 illustrates a top perspective view of a retainer shown in FIG. 15.

FIG. 18 illustrates a bottom perspective view of the retainer shown in FIG. 17.

FIG. 19 illustrates a top perspective view of a disk shown in FIG. 15.

FIG. 20 illustrates a bottom perspective view of a disk according to an embodiment of the present disclosure.

FIG. 21 illustrates a top perspective view of the upper body shown in FIG. 15 with struts extending from a central opening.

FIG. 22 illustrates a perspective view of a crimping device according to an embodiment of the present disclosure.

FIG. 23 illustrates a side cross sectional view of the crimping device shown in FIG. 22.

FIG. 24 illustrates a side cross sectional view of the crimping device shown in FIG. 22.

FIG. 25 illustrates a perspective view of a crimping device according to an embodiment of the present disclosure.

FIG. 26 illustrates a perspective view of a crimping device according to an embodiment of the present disclosure.

FIG. 27 illustrates a perspective view of a crimping device according to an embodiment of the present disclosure.

FIG. 28 illustrates a side cross sectional view of the crimping device shown in FIG. 27 along a center line with an implant and a delivery apparatus present.

FIG. 29 illustrates a perspective view of a crimping device according to an embodiment of the present disclosure.

FIG. 30 illustrates side cross sectional view of a crimping device according to an embodiment of the present disclosure.

FIG. 31 illustrates a perspective view of a crimping device according to an embodiment of the present disclosure.

FIG. 32 illustrates a side cross sectional view of the crimping device shown in FIG. 31.

FIG. 33 illustrates a side cross sectional view of the crimping device shown in FIG. 31 with an implant positioned within the crimping device.

FIG. 34 illustrates a side cross sectional view of the crimping device shown in FIG. 31 compressing the implant.

FIG. 35 illustrates a side cross sectional view of a delivery apparatus according to an embodiment of the present disclosure.

FIG. 36 illustrates a side cross sectional view of a delivery apparatus according to an embodiment of the present disclosure.

FIG. 37 illustrates a side cross sectional view of a delivery apparatus according to an embodiment of the present disclosure.

FIG. 38 illustrates a side cross sectional view of a delivery apparatus according to an embodiment of the present disclosure.

FIG. 39 illustrates a perspective view of a delivery apparatus according to an embodiment of the present disclosure.

FIG. 40 illustrates a perspective view of a delivery apparatus according to an embodiment of the present disclosure.

FIG. 41 illustrates a perspective view of a delivery apparatus according to an embodiment of the present disclosure.

FIG. 42 illustrates a perspective view of a delivery apparatus according to an embodiment of the present disclosure.

FIG. 43 illustrates a side cross sectional view of a delivery apparatus according to an embodiment of the present disclosure.

FIG. 44 illustrates a side cross sectional view of the delivery apparatus shown in FIG. 43 according to an embodiment of the present disclosure.

FIG. 45 illustrates a side cross sectional view of the delivery apparatus shown in FIG. 43 according to an embodiment of the present disclosure.

FIG. 46 illustrates a side cross sectional view of the delivery apparatus shown in FIG. 43 according to an embodiment of the present disclosure.

FIG. 47 illustrates a perspective view of a delivery apparatus capsule according to an embodiment of the present disclosure.

FIG. 48 illustrates a close up cross sectional view of the delivery apparatus along line 48-48 shown in FIG. 47.

FIG. 49 illustrates a perspective view of a delivery apparatus capsule according to an embodiment of the present disclosure.

FIG. 50 illustrates a perspective view of a delivery apparatus capsule according to an embodiment of the present disclosure.

FIG. 51 illustrates a perspective view of a delivery apparatus capsule according to an embodiment of the present disclosure.

FIG. 52 illustrates a cross sectional view of a delivery apparatus capsule according to an embodiment of the present disclosure.

FIG. 53 illustrates a cross sectional view of a delivery apparatus capsule according to an embodiment of the present disclosure.

FIG. 54 illustrates a cross sectional view of a delivery apparatus capsule according to an embodiment of the present disclosure.

FIG. 55 illustrates a side cross sectional view of a delivery apparatus capsule according to an embodiment of the present disclosure.

FIG. 56 illustrates a perspective view of a delivery apparatus capsule shown in FIG. 53 according to an embodiment of the present disclosure.

FIG. 57 illustrates a side view of the delivery apparatus capsule shown in FIG. 56 in a flexed configuration.

FIG. 58 illustrates a side cross sectional view of a delivery apparatus capsule according to an embodiment of the present disclosure.

FIG. 59 illustrates a side perspective view of a portion of the delivery apparatus capsule shown in FIG. 58.

FIG. 60 illustrates a side perspective view of a portion of the delivery apparatus capsule shown in FIG. 58.

FIG. 61 illustrates a side perspective view of an elongate flat braid ribbon according to an embodiment of the present disclosure.

FIG. 62 illustrates a side perspective view of an elongate flat braid ribbon according to an embodiment of the present disclosure.

FIG. 63 illustrates a side perspective view of an elongate flat braid ribbon according to an embodiment of the present disclosure.

FIG. 64 illustrates a cross sectional view of a portion of the delivery apparatus capsule shown in FIG. 58.

FIG. 65 illustrates a cross sectional view of a portion of a delivery apparatus capsule according to an embodiment of the present disclosure.

FIG. 66 illustrates a hypotube cut pattern according to an embodiment of the present disclosure.

FIG. 67 illustrates a hypotube cut pattern according to an embodiment of the present disclosure.

FIG. 68A illustrates a side cross sectional view of a portion of a delivery apparatus according to an embodiment of the present disclosure.

FIG. 68B illustrates a side cross sectional view of the portion of the delivery apparatus shown in FIG. 68A.

FIG. 68C illustrates a side cross sectional view of a portion of the delivery apparatus shown in FIG. 68A displaced from the position shown in FIG. 68A.

FIG. 69 illustrates a perspective view of an outer layer of a delivery apparatus.

FIG. 70 illustrates a perspective view of a middle layer of the delivery apparatus shown in FIG. 69.

FIG. 71A illustrates a perspective view of a rail assembly of the delivery apparatus shown in FIG. 69.

FIG. 71B illustrates a cross sectional view of the rail assembly of the delivery apparatus shown in FIG. 69.

FIG. 72 illustrates a perspective view of an inner layer of the delivery apparatus shown in FIG. 69.

FIG. 73 illustrates a perspective view of a nose cone assembly of the delivery apparatus shown in FIG. 69.

FIG. 74 illustrates a perspective view of a handle of the delivery apparatus shown in FIG. 69.

FIG. 75 illustrates a side cross sectional view of the handle of the delivery apparatus shown in FIG. 74.

### DETAILED DESCRIPTION

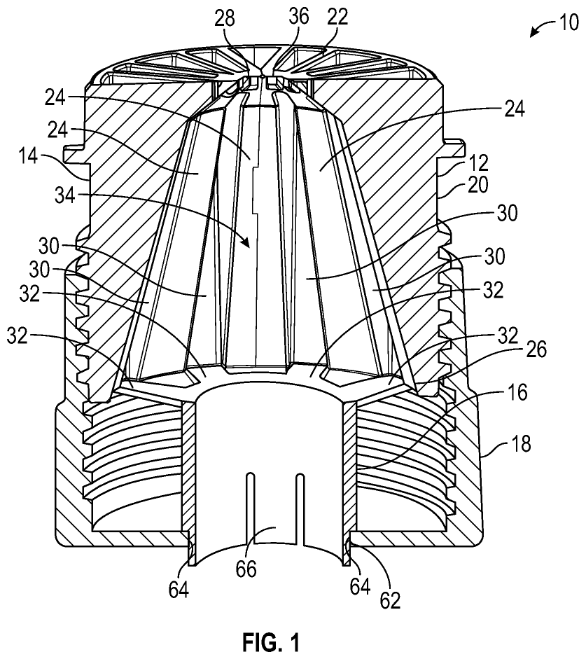

FIG. 1 illustrates a side cross sectional view of a crimping device 10 for an implant. The crimping device 10 may be configured to compress the implant to place the implant in a compressed state for placement within an implant retention area of a delivery apparatus, for delivery to a desired location within a patient's body. Upon being placed at the location within the patient's body, the implant may be released from the implant retention area and deployed to the location within the patient's body in an expanded or uncompressed state.

The implant in embodiments may comprise a medical implant, which may comprise an implant for implantation within a portion of a patient's body. The implant for implantation may comprise a prosthetic implant in embodiments, configured to replace or repair the functioning of a native structure within the patient's body, such as a native heart valve. Other forms of implants may be utilized in embodiments.

The crimping device 10 may include a body 12 having an upper body 14, a pressing body 16, and an actuator 18.

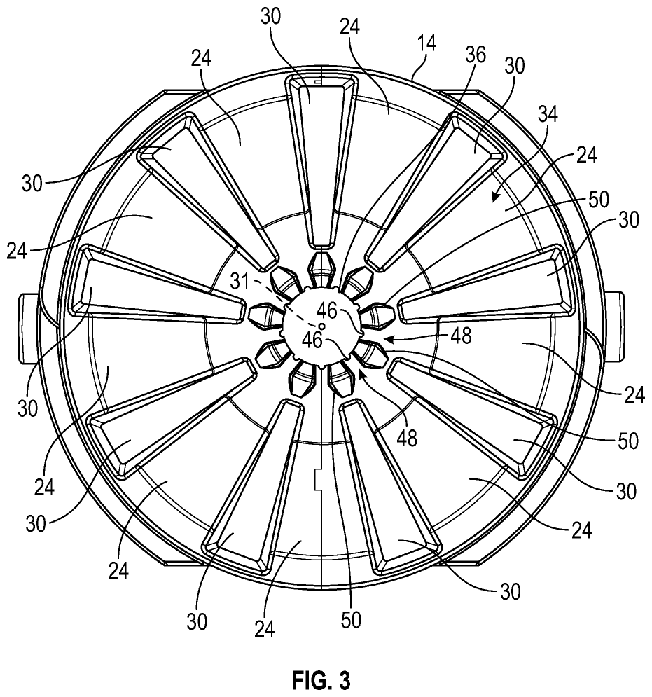

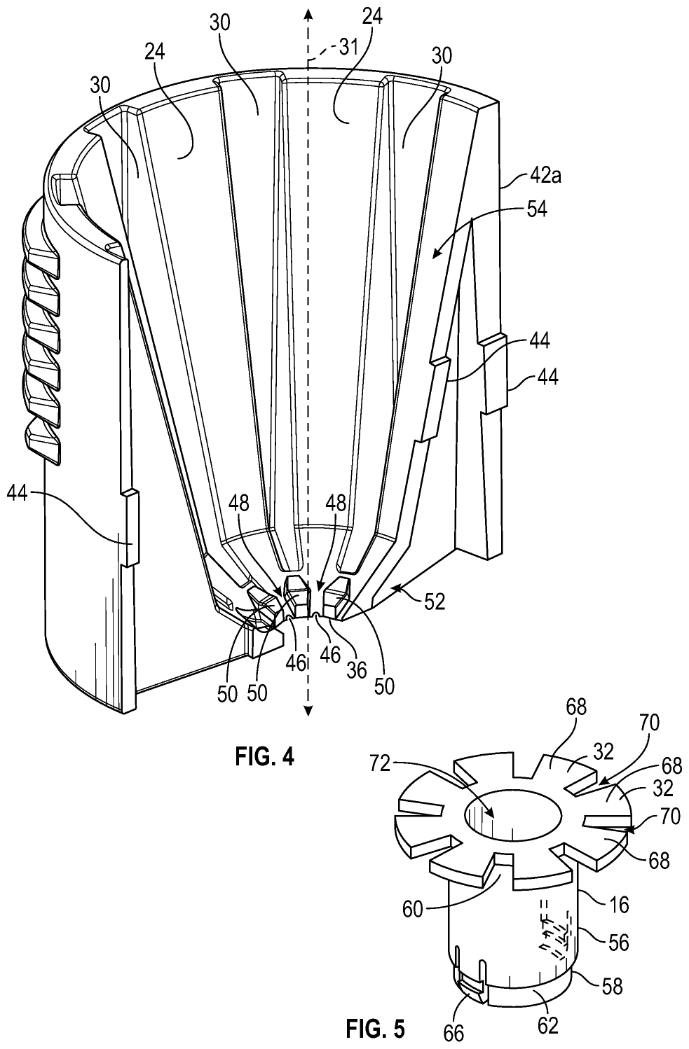

The upper body 14 may include an outer surface 20 and a top surface 22 spanning the outer surface 20. The upper body 14 may further include an inner surface 24 forming a funnel extending from a wide end portion 26 of the funnel to a narrow end portion 28 of the funnel. The inner surface 24 may have a conical shape as shown in FIG. 1 or may have another shape that forms a funnel as desired. The inner surface 24 may be angled relative to the longitudinal axis 31 (marked in FIGS. 3 and 4) of the upper body 14 to form the funnel. The inner surface 24 may surround a cavity 34 for receiving the implant.

The upper body 14 may further include slots 30 extending longitudinally along the longitudinal axis 31 (marked in FIGS. 3 and 4) of the upper body 14 for the pressing surface 32 of the pressing body 16 to slide within, to guide the pressing body 16 within a cavity 34 that is surrounded by the inner surface 24. The pressing surface 32 may include flanges extending radially outward from the pressing body 16 and that fit within the slots 30, to prevent rotation of the pressing body 16 within the cavity 34.

The portions of the inner surface 24 between the slots 30 may be angled to form the funnel.

The top surface 22 of the upper body 14 may include an opening 36. The opening 36 may be configured for a portion of the implant to be accessible at, for example, a portion of the implant may extend through the opening 36 to engage a delivery apparatus or a portion of a delivery apparatus may extend through the opening 36 to engage the implant. The opening 36 may be positioned at the narrow end portion 28 of the funnel. The opening 36 may extend around the longitudinal axis that the upper body 14 surrounds.

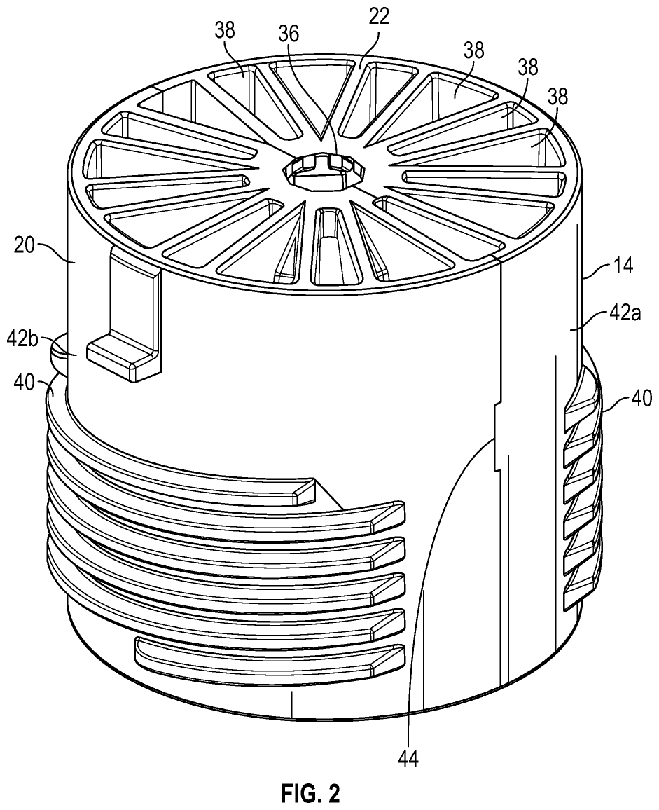

FIG. 2 illustrates a top perspective view of the upper body 14. The top surface 22 of the upper body 14 is shown to include the opening 36, as well as a plurality of additional openings 38 that may be utilized, for example, to vent air out of the cavity 34 shown in FIG. 1 as the implant is pressed in a direction towards the opening 36. The additional openings 38 may extend radially outward from the central opening 36, and may be connected to the slots 30 shown in FIG. 1, to allow air to pass through the slots 30 and through the openings 38 to exit the cavity 34.

The outer surface 20 of the upper body 14 may include threading 40 that may be configured to engage threading 43 (marked in FIGS. 6 and 7) of the actuator 18. The threading 40 may allow the actuator 18 to be rotated relative to the upper body 14 to cause the pressing body shown in FIG. 1 to slide longitudinally within the cavity 34 in directions towards and away from the opening 36.

The upper body 14 may include multiple sides that are brought together to form the upper body 14. For example, as shown in FIG. 2, the upper body 14 includes two opposing sides 42a, 42b that are joined together to form the upper body 14. The sides 42a, 42b may form halves of the upper body 14 as shown in FIG. 2. In other embodiments, a greater number of sides may be utilized (e.g., three, four, etc.), or the upper body 14 may comprise a unitary body in other embodiments. The sides 42a, 42b may be configured to separate from each other along a plane that the longitudinal axis of the upper body 14 passes through. As such, the sides 42a, 42b may be configured to separate from each other to expose the internal cavity 34 shown in FIG. 1 and open the central opening 36. Such a feature may allow the upper body 14 to be opened after the implant has been coupled to a portion of a delivery apparatus, so that the crimping device 10 can be removed from the implant and the delivery apparatus prior to the delivery apparatus being inserted into a portion of a patient's body. The sides 42a, 42b may include a key structure 44 that allows the sides 42a, 42b to couple to each other in the desired orientation. For example, FIG. 4 illustrates the side 42a isolated, with the key structure 44 shown to comprise protrusions that are configured to fit within recesses of the side 42b.

The body 12 may further comprise one or more retainers (e.g., a retainer ring or retainer plate) that are utilized to couple the sides 42a, 42b together, to reduce the possibility of undesired separation of the sides 42a, 42b. The retainer may be removed prior to separation of the sides 42a, 42b.

FIG. 3 illustrates a bottom view of the upper body 14 illustrating the relative position of the opening 36 at the upper end of the cavity 34. The upper body 14 may include a plurality of guide channels having a variety of forms in FIG. 3. The guide channels may each be configured for a portion of the implant to slide in to guide the implant at the opening 36. For example, the guide channels 46 may comprise notches in the surface of the upper body 14. The notches may extend from the inner surface 24 of the upper body 14 to the top surface 22 of the upper body. The notches may be positioned radially about the opening 36 and may be equally spaced from each other. The notches may extend radially outward from the opening 36 and surround the opening 36 and extend in a plane with the opening 36. The number of notches of the upper body 14 may correspond to the number of struts of the implant that end in end tab portions. In other embodiments, a number of notches may be different than the number of struts of the implant that end in end tab portions.

The guide channels of the upper body may further include the guide channels 48 formed between protrusions 50 of the inner surface 24. The guide channels 48 may each be configured for a portion of the implant to slide in to guide the implant at the opening 36. The guide channels 48 may be positioned radially about the opening 36 and may be equally spaced from each other. The guide channels 48 may extend radially outward from the opening 36. The guide channels 48 are positioned on the inner surface 24 of the upper body 14. The guide channels 48 extend at an angle relative to a plane of the opening 36. The guide channels 48 extend longitudinally in a direction from the narrow end portion 28 to the wide end portion 26. The guide channels 48 may be aligned with the notches. The number of guide channels 48 of the upper body 14 may correspond to the number of struts of the implant that end in end tab portions. In other embodiments, a number of guide channels 48 may be different than the number of struts of the implant that end in end tab portions.

FIG. 4 illustrates a side perspective view of the side 42a of the upper body 14, with the opposing side 42b removed from view. The upper body 14 is inverted from the position shown in FIG. 1. The protrusions 50 are shown to comprise bumps on the inner surface 24 of the upper body 14 that are spaced from each other to form the channels 48 between the bumps. The protrusions 50 extend radially outward from the opening 36. The protrusions 50 and the guide channels 48, 46 are positioned proximate the opening 36. The guide channels 46, 48 are positioned at the narrow end portion 28 of the funnel. The protrusions 50 and the guide channels 48, 46 are positioned at a distal portion 52 of the funnel, which may have a greater surface angle relative to the longitudinal axis 31 than a proximal portion 54 of the funnel. The distal portion 52 may be angled towards the opening 36 at a greater angle than the proximal portion 54 of the funnel. The distal portion 52 of the funnel may be proximate the opening 36.

FIG. 5 illustrates a perspective view of the pressing body 16 separate from other components of the crimping device 10. The pressing body 16 may include a support shaft 56 having a lower end portion 58 and an upper end portion 60 at which the pressing surface 32 is positioned. The lower end portion 58 may include a recess 62 that a bearing surface 64 (marked in FIGS. 1 and 6) of the actuator 18 may rotate along and press against to drive the pressing body 16 longitudinally towards and away from the opening 36. The lower end portion 58 may further include a releasable coupler 66 (which may comprise a ledge or another form of releasable coupler) that is configured to extend over a portion of the actuator 18 to secure the pressing body 16 to the bearing surface 64 of the actuator 18.

The pressing surface 32 at the upper end portion 60 of the pressing body 16 may be configured to press against the implant positioned within the cavity 34 (marked in FIG. 1) to compress the implant within the cavity 34 and drive the implant towards the opening 36. The pressing surface 32 may include a plurality of radially extending flanges 68 separated by radially extending gaps 70. The radially extending flanges 68 may extend into the slots 30 shown in FIG. 1 to prevent the pressing body 16 from rotating within the cavity 34. The pressing surface 32 may comprise a flat surface extending transverse to the longitudinal axis 31 of the upper body 14 or may have another configuration in other embodiments. The pressing body 16 may include a central channel 72 configured for portions of the delivery apparatus to pass through.

FIG. 6 illustrates a perspective view of the actuator 18 shown in FIG. 1. The actuator 18 may comprise a ring configured to extend around the outer surface 20 of the upper body 14. The actuator 18 may include an outer surface 74 configured for a user to grip to rotate the actuator 18. For example, the outer surface 74 may include a grip structure such as a plurality of indentations 76 positioned circumferentially about the outer surface 74 to improve the grip of a user upon the outer surface 74. The actuator 18 may include an inner surface 78 facing opposite the outer surface 74 and including threading 43 that is configured to engage the threading 40 (marked in FIG. 2) of the upper body 14. As such, the actuator 18 may rotate relative to the upper body 14, with the engaged threadings 40, 43 causing longitudinal motion of the actuator 18 relative to the upper body 14 and thus driving the pressing body 16 longitudinally along with the actuator 18.

FIG. 7 illustrates a cross sectional view of the actuator 18 shown in FIG. 6. The actuator 18 may include the bottom bearing surface 64 that is configured to engage the recess 62 of the pressing body 16 to allow the actuator 18 to rotate relative to the pressing body 16 and drive the pressing body 16 longitudinally. The actuator 18 may further include an opening 80 configured for portions of the delivery apparatus to pass through.

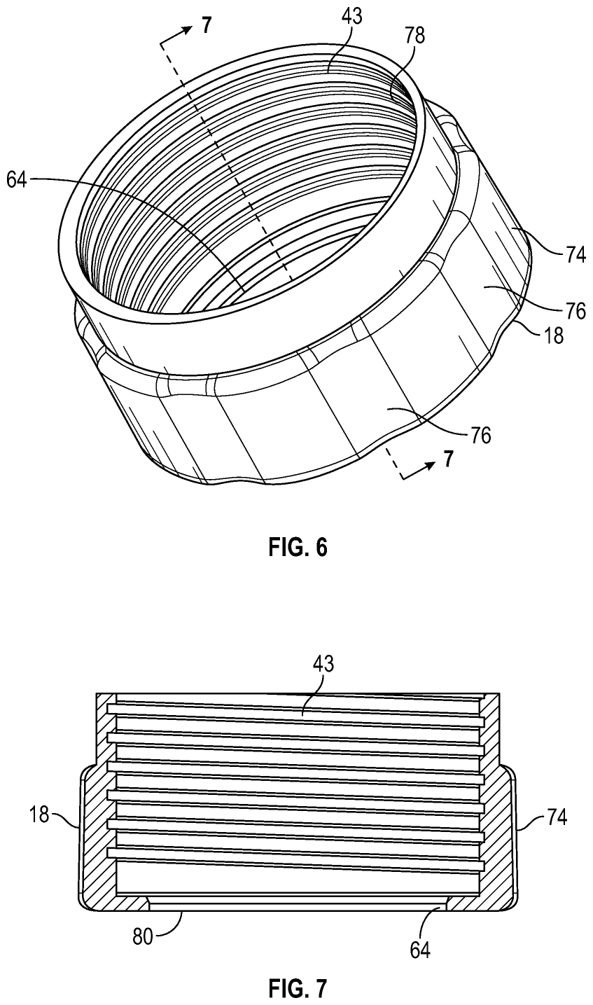

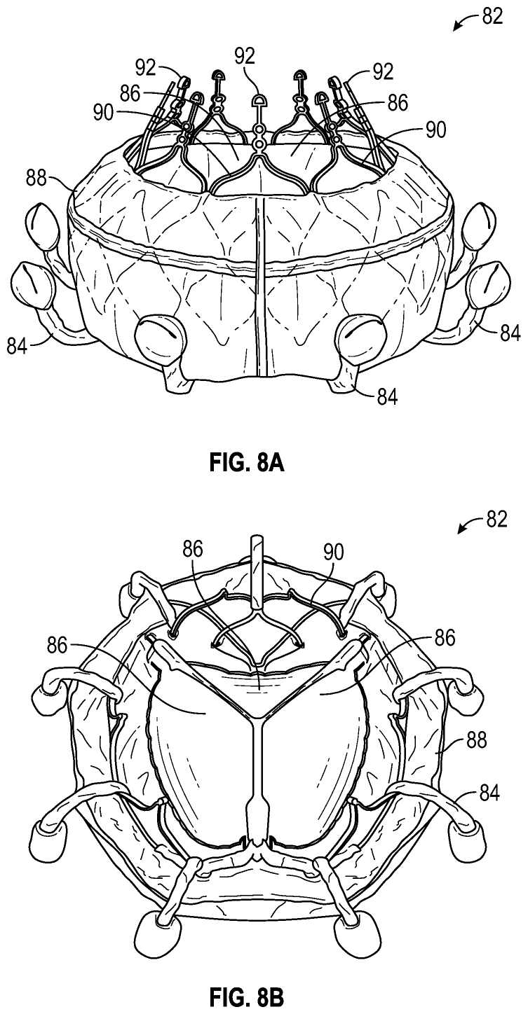

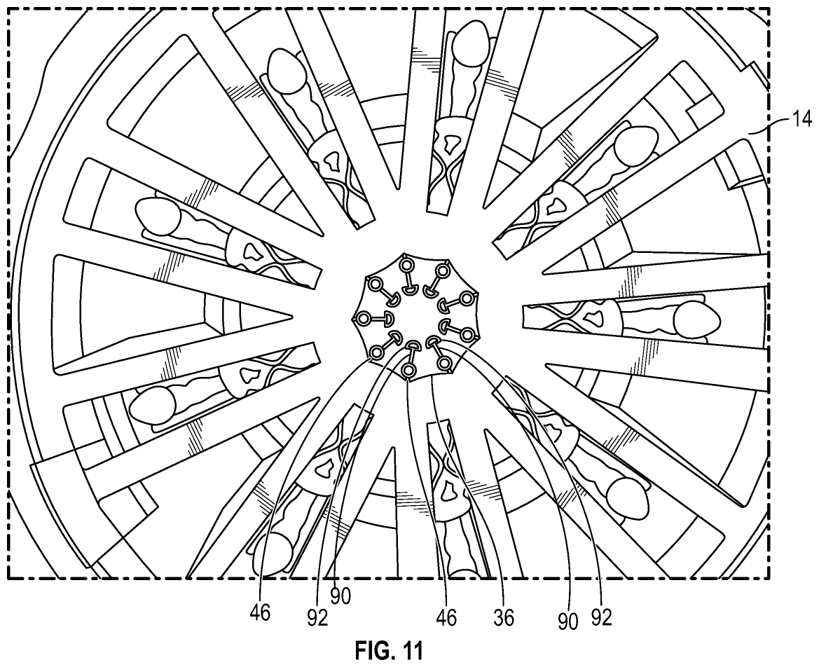

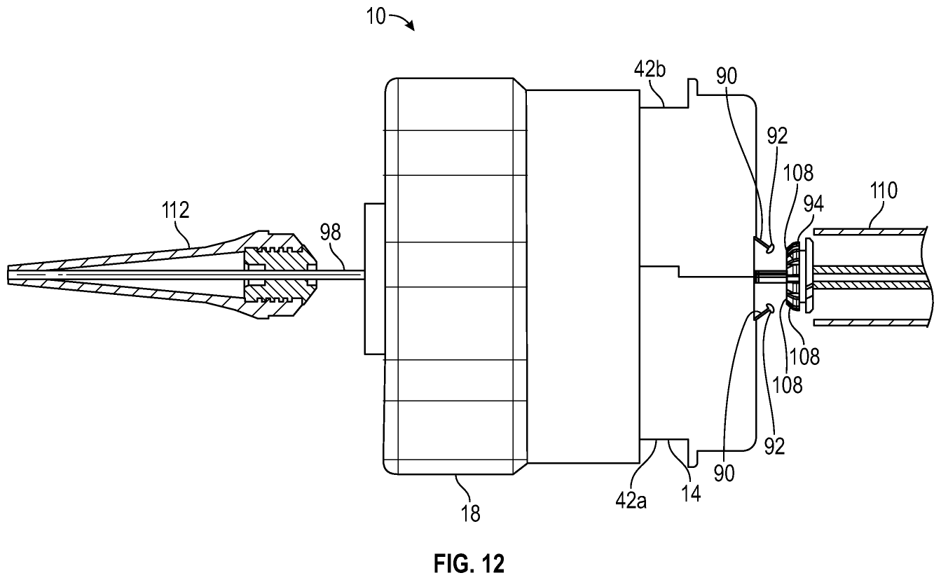

The crimping device 10 may be configured to compress an implant, which may be a medical implant such as a prosthetic valve, and which may further comprise a prosthetic heart valve. FIG. 8A for example, illustrates a perspective view of an implant 82 that may be utilized with the systems, apparatuses, and methods disclosed herein. The implant 82 comprises a prosthetic heart valve, which may include distal anchors 84, prosthetic valve leaflets 86 (more clearly shown in the bottom view of FIG. 8B), and a skirt 88 extending around the prosthetic valve leaflets 86. Further, the implant may include struts 90 that form the frame of the implant 82 and certain of those struts end in end tab portions 92 that are configured to couple to a portion of a delivery apparatus. For example, the end tab portions 92 may be configured to engage a coupler 94 (as marked in FIG. 12) of a delivery apparatus to couple the implant 82 to the delivery apparatus. The end tab portions 92 may include flared half-dome shapes, or another shape as desired to couple to the coupler 94. FIG. 8B illustrates a bottom view of the implant 82.

The implant 82 shown in FIGS. 8A and 8B is configured as a prosthetic mitral valve, however, other forms of implants may be utilized as desired. For example, prosthetic aortic, tricuspid, or pulmonary valves may be utilized with the systems, apparatuses, and methods disclosed herein. Further, other forms of implants such as stents or other implants may be utilized with the systems, apparatuses, and methods disclosed herein as well. The implant 82 shown in FIGS. 8A and 8B may utilize the distal anchors 84 to engage a ventricular side of a mitral valve. In embodiments, the implant 82 may include proximal anchors that may engage an atrial side of a mitral valve. Other forms of anchors may be utilized, as well as other forms of implants may be utilized in other embodiments.

Features of an implant that may be utilized are disclosed in U.S. patent application Ser. No. 16/028,172, filed on Jul. 5, 2018, and published as U.S. Patent Publication No. 2019/0008640, the entire contents of which are incorporated by reference herein. Additional details and example designs for an implant and prosthesis that may be utilized in embodiments herein are described in U.S. Pat. Nos. 8,403,983, 8,414,644, 8,652,203 and U.S. Patent Publication Nos. 2011/0313515, 2012/0215303, 2014/0277390, 2014/0277422, 2014/0277427, 2018/0021129, and 2018/0055629, the entirety of these patents and publications are hereby incorporated by reference and made a part of this specification. Further details and embodiments of a replacement heart valve or prosthesis and its method of implantation are described in U.S. Publication Nos. 2015/0328000 and 2016/0317301 the entirety of each of which is hereby incorporated by reference and made a part of this specification.

The implant 82 is compressible, such that the implant 82 may be compressed in a direction radially towards a longitudinal axis that the implant 82 surrounds. For example, the frame of the implant 82 may be flexible, to allow for compression of the implant 82. Upon compression in the direction radially towards the longitudinal axis, the implant 82 may increase in length longitudinally along the longitudinal axis (with the end tab portions 92 and the distal anchors 84 extending in opposite directions along the longitudinal axis, for example as shown in FIG. 36). In certain embodiments, the implant 82 may be configured to compress in the direction radially towards the longitudinal axis without the implant 82 increasing in length.

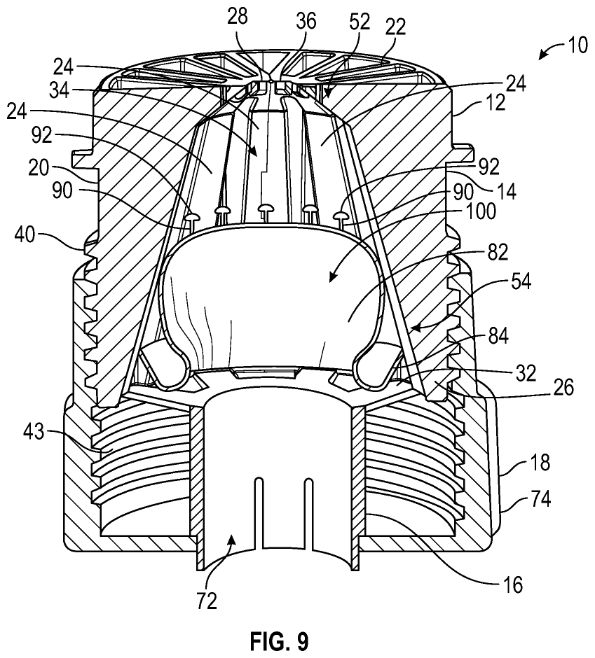

FIG. 9 illustrates the implant 82 in position within the cavity 34 of the body 12. Certain features, such as the valve leaflets of the implant and features of the frame and skirt are not depicted for clarity.

In operation, the actuator 18 may first be uncoupled from the upper body 14 by being unscrewed from the upper body 14. Further, in an embodiment in which a portion of the delivery apparatus (such as an elongate shaft 98 as shown in FIG. 12) is to pass through the central cavity 34 of the body 12, the sides 42a, b of the upper body 14 may be separated and then assembled together upon the portion of the delivery apparatus. The implant 82 may then be inserted into the cavity 34 of the body 12 with the portion of the delivery apparatus extending through a central channel 100 of the implant. The actuator 18 may then be coupled to the upper body 14 with the portion of the delivery apparatus passing through the central channel 72 of the pressing body 16. However, in an embodiment in which a portion of the delivery apparatus is not positioned within the central cavity 34, then the actuator 18 may first be uncoupled from the upper body 14 by being unscrewed from the upper body 14, and the implant 82 may be inserted into the cavity 34. The actuator 18 may then be coupled to the upper body 14. Other methods of inserting the implant 82 into the cavity 34 may be utilized as desired.

FIG. 9 illustrates that the upper struts 90 of the implant 82 extend in a direction towards the opening 36 upon insertion into the cavity 34. The end couplers or end tab portions 92 of the implant accordingly extend in a direction towards the opening 36. The distal anchors 84 of the implant 82 rest upon the pressing surface 32 of the pressing body 16. The implant 82 is positioned within the cavity 34 such that the inner surface 24 of the upper body 14 contacts an outer surface of the implant 82.

The crimping device 10 may be operated such that the outer surface 20 of the upper body 14 is gripped by a user, as well as the outer surface 74 of the actuator 18. The outer surface 74 of the actuator 18 may be rotated relative to the outer surface 20 of the upper body 14, to longitudinally advance the actuator 18 along the upper body 14 due to the engagement of the threading 40, 43. The rotation of the actuator 18 drives the pressing body 16 in a direction towards the opening 36, which accordingly causes the implant 82 to be moved towards the opening 36 due to the force applied by the pressing surface 32 to the implant 82.

The inner surface 24 of the upper body 14 contacts the implant 82, to cause the end tab portions 92 to be drawn nearer towards each other in a direction towards the longitudinal axis of the upper body 14 as the implant 82 is advanced towards the opening 36. The implant 82 is slid within the cavity 34 of the funnel in a direction from the wide end portion 26 to the narrow end portion 28. The conical shape of the funnel formed by the inner surface 24 causes the end tab portions 92 to be drawn progressively nearer towards each other as the implant 82 advances towards the opening 36. Upon the end tab portions 92 reaching the distal portion 52 of the upper body 14, the end tab portions 92 may deflect to a greater amount than in the proximal portion 54 of the upper body 14 due to the greater angle of the distal portion 52 relative to the longitudinal axis of the upper body 14. The implant 82 may be progressively further compressed as the implant 82 advances towards the opening 36 due to the conical shape of the funnel formed by the inner surface 24.

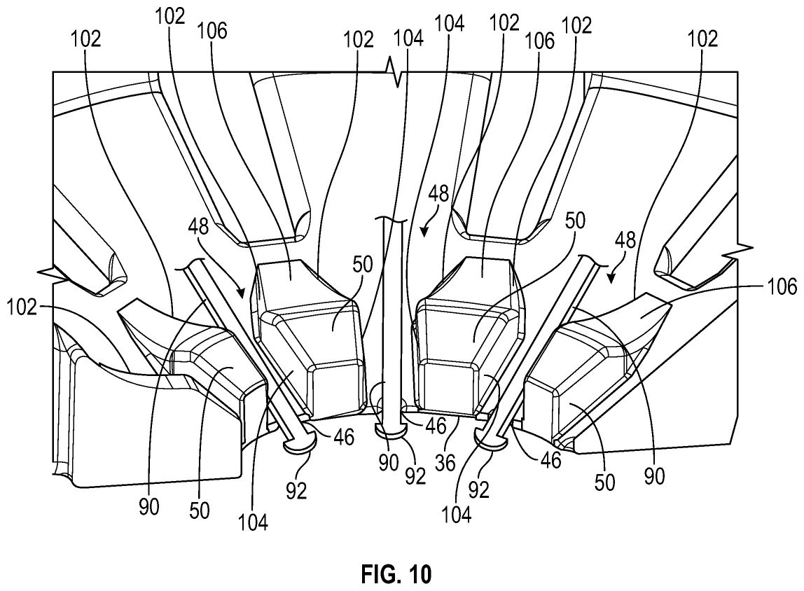

Upon the end tab portions 92 approaching the opening 36, the struts 90 may extend within and slide within the guide channels 46, 48. FIG. 10 for example, illustrates the struts 90 extending within and sliding within both the guide channels 46, 48. The struts 90 and end tab portions 92 are oriented at the opening 36.

With regard to the guide channels 48, the struts 90 may be deflected into the respective guide channel 48 by angled portions 102 of the protrusions 50 that angle in a direction towards each other and towards the guide channel 48 in a direction towards the opening 36. As such, the struts 90 may contact the angled portions 102 to deflect into the respective guide channel 48. Further, the protrusions 50 may include linear side walls 104 that extend parallel with the linear side walls 104 of the adjacent protrusion 50 to form a guide channel 48 having a consistent width for the strut 90 to extend in. In other embodiments, the width of the guide channel 48 may vary along the length of the guide channel 48. The protrusions 50 may further include angled upper surfaces 106 that may angle in a direction away from the inner surface 24 as the angled upper surfaces 106 approach the opening 36. Thus, a skirt or other material positioned between the struts 90 may be deflected away from the struts 90 and the opening 36 as the struts 90 are advanced toward the opening 36. Such a feature may reduce the possibility of the skirt or other material interfering with the end tab portions 92 at the opening 36.

With regard to the guide channels 46, the struts 90 may pass through the notches of the guide channels 46 to be guided at the opening 36.

The equal spacing of the guide channels 46, 48 may guide the struts 90 so that the struts 90 are equally spaced from each other at the opening 36. Thus, the guide channels 46, 48 may guide the struts 90 to be positioned at an equal spacing from each other at the opening 36 such that the end tab portions 92 may be more easily coupled to a coupler 94 (as marked in FIG. 12) of a delivery apparatus. The coupler 94 (as marked in FIG. 12) may include receiving portions 108 configured to engage the end tab portions 92. The receiving portions 108 may be equally spaced from each other and thus the equal spacing of the end tab portions 92 may improve the engagement between the end tab portions 92 and the receiving portions 108. The spacing of the end tab portions 92 produced by the guide channels 46, 48 may be the same spacing that the receiving portions 108 have relative to each other. Such a feature may allow for ease of alignment between the end tab portions 92 and the receiving portions 108. In other embodiments, other spacing of the end tab portions 92 may be produced by the guide channels 46, 48.

FIG. 11 illustrates a top view of the upper body 14 showing the end tab portions 92 guided at the opening 36 by the guide channels 46, 48. An end portion of the implant has been passed through the opening 36. The end tab portions 92 extend towards each other and protrude from the opening 36. The end tab portions 92 are equally spaced from each other about the longitudinal axis of the upper body 14 and are configured for engagement with a coupler 94 (as marked in FIG. 12) of a delivery apparatus. Without the presence of the guide channels 46, 48, the end tab portions 92 may not be positioned correctly at the opening 36 and may be bunched together or otherwise in an undesired orientation.

FIG. 12 illustrates a side view of the crimping device 10 in position around an elongate shaft 98 of a delivery apparatus. The crimping device 10 advances the end tab portions 92 out of the opening 36 (marked in FIG. 11) such that the end tab portions 92 extend from the opening 36. A fewer number of end tab portions 92 are shown in FIG. 12 than in FIG. 11 for clarity. The end tab portions 92, guided by the guide channels 46, 48, may accordingly be coupled to the receiving portions 108 of the coupler 94. In such a configuration, the end tab portions 92 may be secured to the coupler 94. For example, a retaining ring or a portion of the capsule 110 may be passed over the coupler 94 to retain the end tab portions 92 to the coupler 94.

The crimping device 10 may continue to operate to compress the implant 82 by the actuator 18 continuing to advance the pressing body 16 towards the opening 36. A portion, or the entirety of the implant 82, may be passed through the opening 36 by being advanced by the pressing body 16. The capsule 110 may be advanced over the implant 82 to retain the implant 82 within an implant retention area of the capsule 110 as the implant 82 is passed through the opening 36. The crimping device 10 may then be disassembled to allow the crimping device 10 to be removed from around the elongate shaft 98 of the delivery apparatus. For example, the actuator 18 may be rotated in a reverse direction to unscrew and separate the actuator 18 from the upper body 14. Further, the pressing body 16 may be separated from the upper body 14. The upper body 14 may then be separated into the sides 42a, 42b to open the opening 36 and the cavity 34 shown in FIG. 1 and allow the sides 42a, 42b to be removed from the elongate shaft 98 of the delivery apparatus.

In certain embodiments, upon the implant 82 being coupled to the coupler 94, the crimping device 10 may be disassembled. The implant 82 may then be further compressed by separate crimping devices or funnel structures, or other devices, to fit within the capsule 110.

In certain embodiments, at least a portion of the delivery apparatus may pass through the opening 36 (marked in FIG. 1) to couple to the end tab portions 92. For example, the coupler 94 may be inserted through the opening 36 to couple to the end tab portions 92, which may be guided by the guide channels 46, 48 in a similar manner as disclosed herein. The end tab portions 92 in such an embodiment may not pass through the opening 36.

In certain embodiments, the crimping device 10 may not extend over a portion of the delivery apparatus. For example, the elongate shaft 98 may not be utilized in an embodiment in which a nose cone 112 is not utilized. The crimping device 10 may be moved to the portion of the delivery apparatus that includes the coupler 94 without extending over a portion of the delivery apparatus.

Upon the implant being compressed and positioned within the capsule 110, the implant may have a compressed configuration as shown with the implant in FIG. 36 for example.

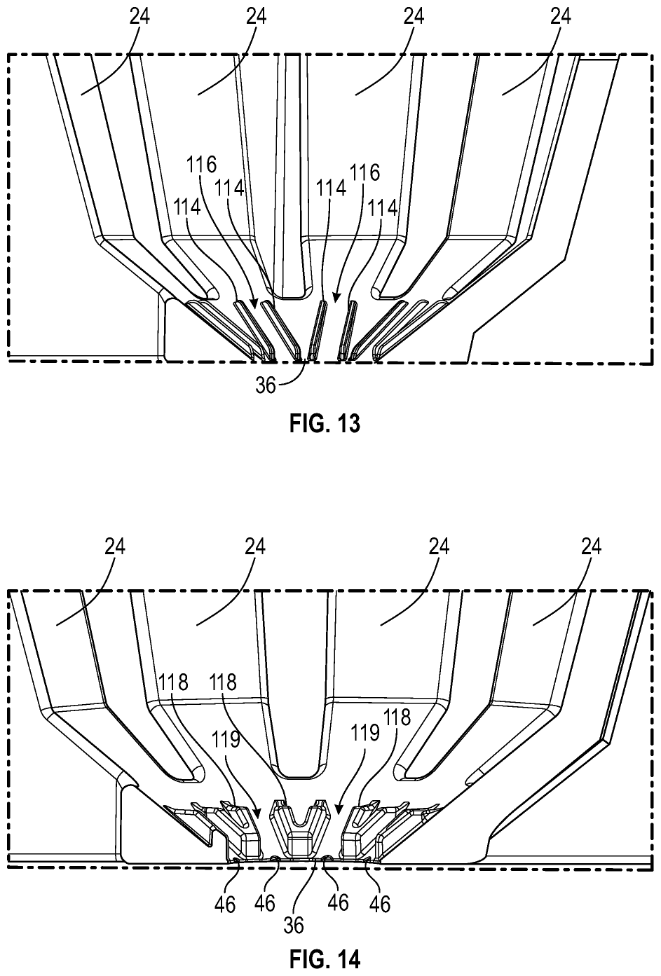

FIG. 13 illustrates an embodiment of the crimping device including protrusions having a different configuration than shown in FIG. 10. The protrusions in the embodiment of FIG. 13 comprise ribs 114 that extend radially outward from the opening 36. The space between the ribs 114 forms the guide channels 116 configured for a portion of the implant to slide in to guide the implant at the opening 36, in a similar manner as discussed above regarding the guide channels 48. The guide channels 46 shown in FIG. 10 may or may not be utilized in such an embodiment.

FIG. 14 illustrates an embodiment of the crimping device including protrusions having a different configuration than shown in FIG. 10. The protrusions in the embodiment of FIG. 14 comprise v-shaped protrusions 118 having an apex facing towards the opening 36. The space between the v-shaped protrusions 118 may form the guide channels 119 configured for a portion of the implant to slide in to guide the implant at the opening 36, in a similar manner as discussed above regarding the guide channels 48. The guide channels 46 are shown in use in FIG. 14, but may or may not be utilized in such an embodiment.

FIG. 15 illustrates a cross sectional view of an embodiment of a crimping device 120 including a body including a disk 124 that includes guide channels that are configured for a portion of the implant to slide in to guide the implant at the opening 126. The crimping device 120, similar to the crimping device 10, may include an upper body 128 having an inner surface 130 forming a funnel for the implant to extend in. The funnel may extend from a wide end portion to a narrow end portion of the funnel including the opening 126. A proximal portion 132 of the upper body 128 may include an inner surface 130 in the form of ribs, and a distal portion 134 of the upper body 128 may include an inner surface that is smooth and has a greater angle of surface relative to the opening 126 than the proximal portion 132 (similar to the distal portion 52 shown in FIG. 4). The inner surface 130 may surround a cavity 168 for receiving the implant.

The disk 124 may be positioned at an upper end portion of the upper body 128, within a recess 136 of an upper surface 138 of the upper body 128. The guide channels of the disk 124 are positioned proximate the opening 126.

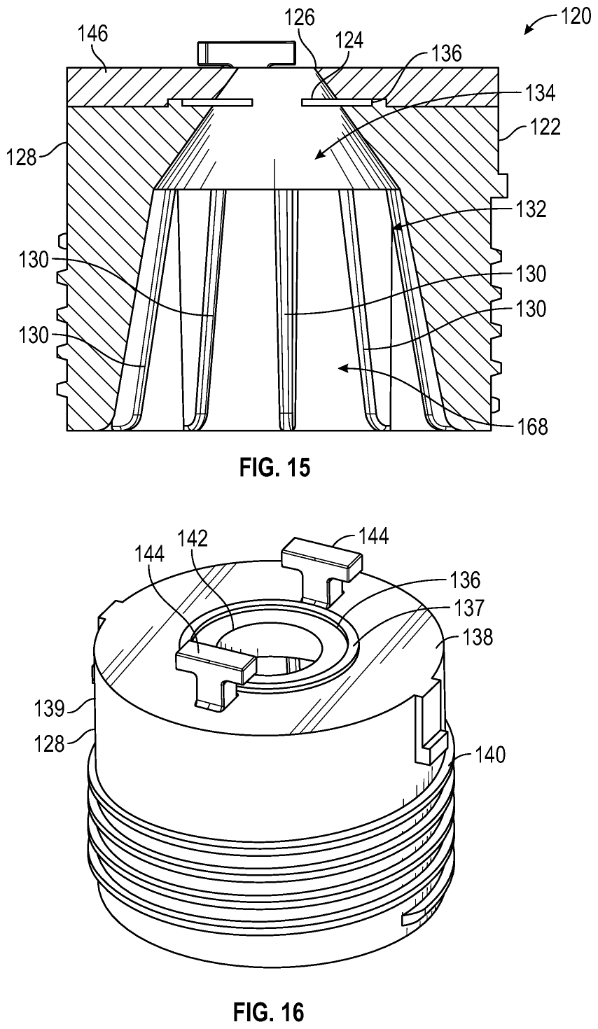

FIG. 16 illustrates a top perspective view of the upper body 128 showing the recess 136 for retaining the disk 124. The recess 136 may surround an opening 142 for a portion of the implant 82 to pass through. The disk 124 may extend over and overlap the opening 142. Further, an outer surface 139 of the upper body 128 includes threading 140 for engaging threading of an actuator 18, in a similar manner as with the crimping device 10. The crimping device 120 may operate in a similar manner as the crimping device 10, with a pressing body 16 within the interior cavity 168 that presses an implant 82 towards the opening 126. The upper body 128 is shown as a unitary body, however in other embodiments the upper body 128 may include separable sides, in a similar manner as discussed regarding the upper body 14.

The upper body 128 may include coupling bodies 144 configured to couple with a retainer 146 for retaining the disk 124 to the upper body 128. The coupling bodies 144 may comprise protrusions that extend upward from the upper surface 138 and may have a “T” shape as shown in FIG. 16. The coupling bodies 144 may be configured to pass through openings of a retainer 146 to couple the retainer 146 to the upper body 128.

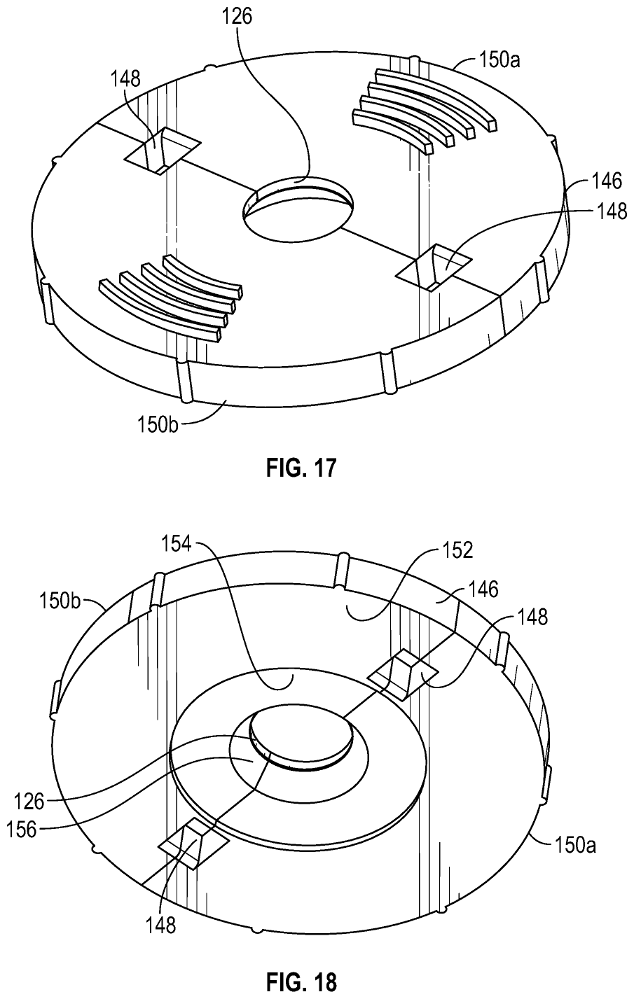

FIG. 17 illustrates a top perspective view of the retainer 146 in the form of a plate that is configured to be positioned on the upper surface 138 of the upper body 128. The retainer 146 may include openings 148 that the coupling bodies 144 of the upper body 128 shown in FIG. 16 are configured to pass through. The retainer 146 may include multiple portions, including two portions 150a, b that are configured to be joined together to sandwich the coupling body 144 within the openings 148 and may be separated to release the coupling body 144 from the openings 148. The retainer 146 may further include the opening 126 for a portion of the implant to be accessible at.

FIG. 18 illustrates a bottom perspective view of the retainer 146. A bottom surface 152 of the retainer 146 may include a recess 154 configured to receive a raised portion 137 of the upper surface 138 marked in FIG. 16 that surrounds the recess 136. The recess 154 may be configured to retain the disk 124. The disk 124 may be sandwiched between the bottom surface 152 of the retainer 146 and the upper surface 138 of the upper body 128. A portion of the retainer 146 may include an inner surface 156 forming a funnel that angles towards the opening 126. The inner surface 156 may be positioned above the disk 124 after assembly of the crimping device 120.

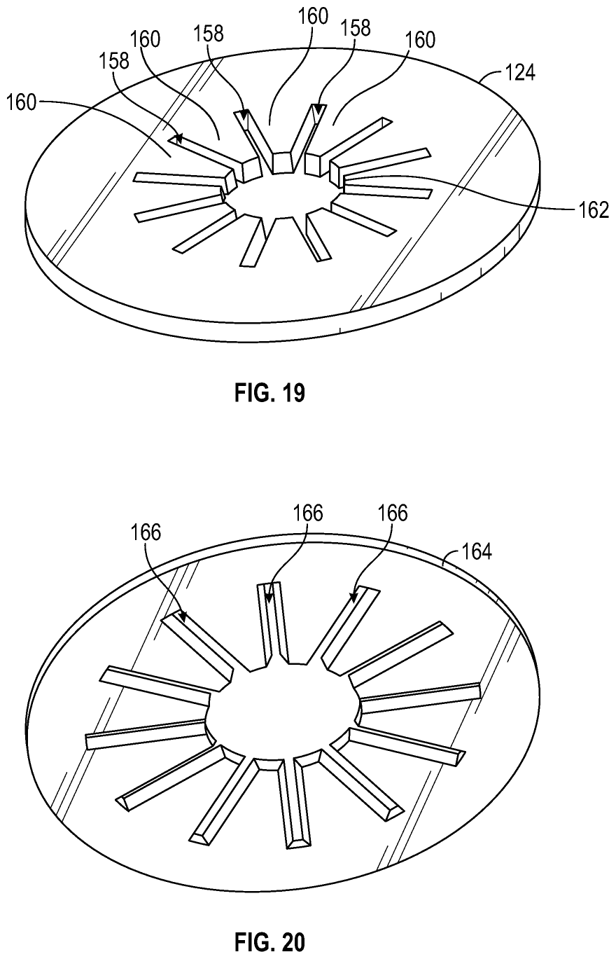

FIG. 19 illustrates a top perspective view of the disk 124. The disk 124 may include a plurality of channels 158 that are configured for a portion of the implant to slide in to guide the implant at the opening 126 shown in FIG. 17. The channels 158 are positioned on an inner portion of a disk 124. The channels 158 may be formed between fins 160 and the fins 160 and channels 158 may both extend radially outward from an opening 162 of the disk 124. The fins 160 and channels 158 may both extend transverse or perpendicular as shown in FIG. 19 to the longitudinal axis that the upper body 128 shown in FIG. 15 extends along.

The disk 124 may be a unitary body as shown in FIG. 19, and may be made of a flexible material. The flexible material may allow the disk 124 to be stretched over a portion of the delivery apparatus during assembly or disassembly of the crimping device 120 upon the portion of the delivery apparatus. Thus, the upper body 128 shown in FIG. 15 may be separated into sides, the retainer 146 shown in FIG. 17 may be separated into sides comprising the portions 150a, b. The upper body 128 may include at least two opposing sides configured to separate from each other to open the cavity 168. The disk 124 shown in FIG. 19 may be stretched to allow for assembly or disassembly of the crimping device 120 upon the portion of the delivery apparatus. In other embodiments, the disk 124 may be separated into sides to allow for assembly or disassembly of the crimping device 120.

The width of each of the guide channels 158 may be constant as shown in FIG. 19, or in embodiments the width may vary. FIG. 20 for example, illustrates a bottom perspective view of a disk 164 including guide channels 166 that are angled. The shape of the edges of the fins may be angled to form the angled shape of the guide channels 166. The angle may be such that the size of the guide channels 166 decreases in a direction towards the opening 126 shown in FIG. 17. Such a feature may enhance the ability of the guide channels 166 to guide the implant at the opening 126.

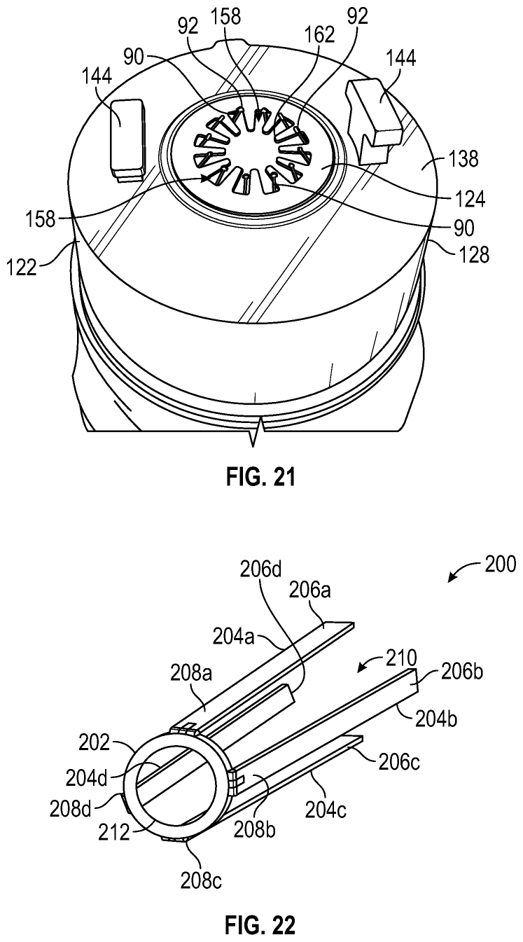

FIG. 21 illustrates a top perspective view of the upper body 122 with the disk 124 in position upon the upper body 122 and the retainer 146 removed for purposes of clarity. The implant 82 may be inserted into the cavity 168 of the upper body 122 in a similar manner as shown in FIG. 9. A pressing body 16 and actuator 18 may then be coupled to the upper body 122 in a similar manner as shown in FIG. 9. The actuator 18 may be configured to move the pressing body 16 to press the implant in a direction towards the opening 126 in a similar manner as shown in FIG. 9. Although not shown, the retainer 146 may be utilized to secure the disk 124 in position on the upper body 122. The crimping device 120 may be positioned upon a portion of a delivery apparatus as shown in FIG. 12, or may not be as desired.

The actuator 18 may be operated to press the implant 82 through the cavity 168 of the upper body 122 towards the opening 126 marked in FIG. 15. As shown in FIG. 21, the guide channels 158 of the disk 124 may have the struts 90 of the implant 82 slide within the channels 158 to guide the implant at the opening 126 marked in FIG. 15. The guide channels 158 of the disk 124 may each configured to space the struts equally at the opening 126. The end tab portions 92 of the implant 82 may then be coupled to a coupler 94 of a delivery apparatus in a similar manner as discussed regarding FIG. 12. The crimping device may then be disassembled if desired, with the sides of the retainer 146 shown in FIG. 17 separated and sides of the upper body 128 being separated in an embodiment in which the upper body 128 includes separable sides. The disk 124, as discussed, may be flexible to allow the disk 124 to be stretched to be slid off of a portion of the delivery apparatus. The actuator 18 may be unscrewed from the upper body 128 and the pressing body 16 may be withdrawn from the cavity 168 shown in FIG. 15 during disassembly.

The configuration of the crimping devices 10, 120 may be varied in other embodiments. The positional terms upper, top, bottom, side, and the like are utilized in a non-limiting manner and are utilized to indicate positional relationships shown in the figures for crimping devices 10, 120. A top surface may remain a top surface although the crimping devices 10, 120 may be rotated to an upside down position, as an example.

The embodiments of crimping devices disclosed in regard to FIGS. 1-21 may be utilized solely, or in combination with features of other systems, apparatuses, or methods disclosed herein.

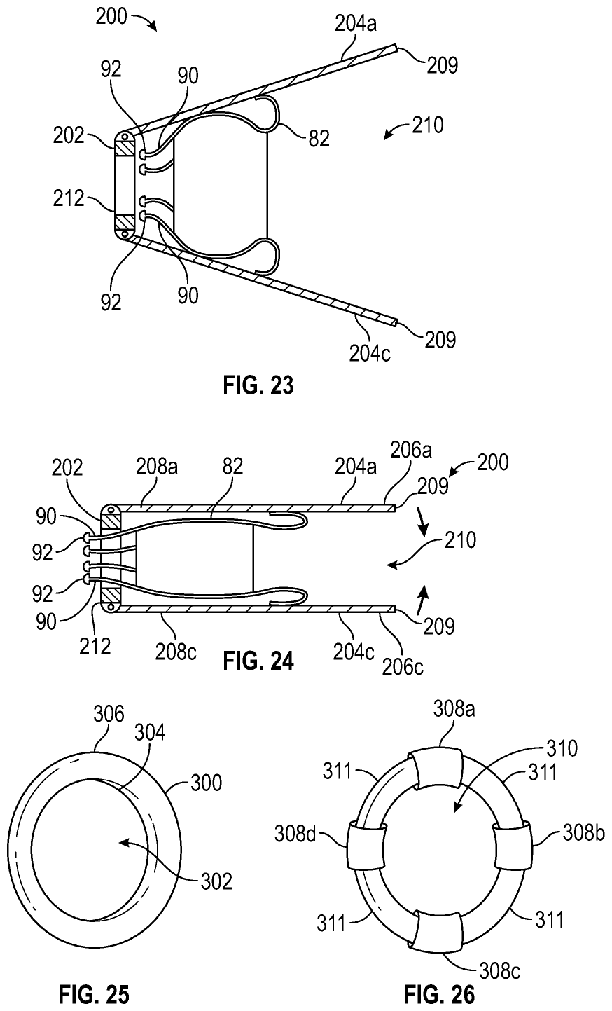

FIG. 22 illustrates an embodiment of a crimping device 200 including a coupler 202 and a plurality of elongate lever arms 204a-d pivotally coupled to the coupler 202. Each of the plurality of elongate lever arms 204a-d may include a proximal end portion 206a-d and a distal end portion 208a-d that is pivotally coupled to the coupler 202. Each of the plurality of elongate lever arms 204a-d is configured to pivot about the coupler 202 from an expanded state (as shown in FIG. 23) in which the plurality of elongate lever arms 204a-d surround an implant receiving region 210, to a reduced state (as shown in FIG. 24) in which the proximal end portions 206a-d are moved closer to each other to compress an implant 82 within the implant receiving region 210. The proximal end portions 206a-d of the plurality of lever arms 204a-d defines an opening 209 marked in FIGS. 23 and 24 for insertion of the implant into the implant receiving region 210.

The coupler 202 as shown in FIG. 22 may comprise a ring having pivot structures that couple to the distal end portions 208a-d of the elongate lever arms 204a-d. The coupler 202 may include a central opening 212 that is configured for a portion of the implant 82 to be passed through. In other embodiments, the coupler 202 may have other forms. For example, the coupler 202 may comprise a pin or other form of pivot structure for directly coupling the distal end portions 208a-d of the elongate lever arms 204a-d together to allow the elongate lever arms 204a-d to rotate relative to each other. In other embodiments, the coupler 202 may have other forms, for example, the opening 212 may not be present and another method of accessing the implant 82 within the implant receiving region 210 may be utilized.

The elongate lever arms 204a-d may comprise stiff lever arms configured to apply a compressive force to the implant 82 within the implant receiving region 210. The proximal end portions 206a-d of the elongate lever arms 204a-d may comprise handles configured to be gripped to move the plurality of elongate lever arms 204a-d to the reduced state. Although four elongate lever arms 204a-d are shown in FIG. 22, a greater (e.g., 5, 6, 7) or lesser number (e.g., 3, 2) of elongate lever arms may be utilized as desired. The four elongate lever arms 204a-d may be spaced equally from each other and the pairs of lever arms 204a, c and 204b, d may pivot within a same plane, and the adjacent lever arms (for example arms 204a, b) may pivot within transverse planes. In embodiments, at least three elongate lever arms may be utilized. In embodiments, at least two elongate lever arms may be utilized.

FIG. 23 illustrates a side cross sectional view of the crimping device 200 shown in FIG. 22. The elongate lever arms 204a-d are shown in an expanded state in which the plurality of elongate lever arms 204a-d surround an implant receiving region 210. The implant 82 is shown positioned within the implant receiving region 210 and positioned between inner surfaces of the elongate lever arms 204a-d. The inner surfaces may comprise pressing surfaces that face towards the implant receiving region 210 and are configured to press against the implant 82. The implant 82 may be in an expanded or uncompressed state. The implant 82 may be inserted into the implant receiving region 210 through the opening 209 defined by the proximal end portions 206a-d of the elongate lever arms 204a-d. As shown, each of the elongate lever arms 204a-d may have a length that is greater than a length of the implant 82, to allow the implant 82 to have its length expand along its longitudinal axis if necessary. The end tab portions 92 of the implant 82 may face towards the opening 212. The length of each of the elongate lever arms may be greater than a length of the implant 82 when compressed, as shown in FIG. 24. The length of the implant 82 as shown in FIG. 24 has increased in the implant receiving region 210.

FIG. 24 illustrates a side cross sectional view of the crimping device 200 in which the elongate lever arms 204a-d have been pivoted to the reduced state. The proximal end portions 206a-d of the elongate lever arms 204a-d may be gripped and rotated. The proximal end portions 206a-d of the elongate lever arms 204a-d have been moved closer to each other to compress the implant 82 within the implant receiving region 210 with the elongate lever arms 204a-d in the reduced state.

A portion of the implant 82, particularly the struts 90 and end tab portions 92 may extend through the opening 212 at the distal end portions 208a-d of the elongate lever arms 204a-d. The opening 212 may be configured for a portion of the implant 82 to be passed through with the plurality of elongate lever arms 204a-d in the reduced state. The compression of the implant 82 may cause the struts 90 and end tab portions 92 to extend through the opening 212 by sliding through the opening 212. The struts 90 and end tab portions 92 may then be coupled to a coupler of a delivery apparatus, for example, a coupler 94 as shown in FIG. 12 while the implant 82 is compressed. The remainder of the implant 82 may then be passed through the opening 212 to be inserted into a capsule 110 as shown in FIG. 12 by being slid within the implant receiving region 210 and through the opening 212. In other embodiments, the crimping device 200 may be configured to be disassembled or otherwise have separable portions to allow the capsule 110 to extend over the remainder of the implant 82. In certain embodiments, a portion of the implant 82 may not extend through the opening 212 upon compression of the implant 82, but a coupler may be inserted to the opening 212 to couple to the implant 82. For example, the coupler may be slid through the opening 212 in a proximal direction. In other embodiments, a coupler may be inserted through the opening 209 formed at the proximal end portions 206a, 206c of the elongate lever arms 204a-d to couple to the implant 82. In other embodiments, a portion of the implant 82 may extend through the opening 209 formed at the proximal end portions 206a, 206c of the elongate lever arms 204a-d to couple to a coupler.

The configuration of the crimping device 200 may be varied in other embodiments.

The embodiments of crimping devices disclosed in regard to FIGS. 22-24 may be utilized solely, or in combination with features of other systems, apparatuses, or methods disclosed herein.

FIG. 25 illustrates an embodiment of a roller 300 that may be utilized as a crimping device for an implant. The roller 300 may surround a channel 302 and may be configured to press against the implant and roll as the implant passes through the channel 302. The rolling motion of the roller 300 may provide a mechanical advantage for compressing the implant within the channel 302. The roller 300 as shown in FIG. 25 may comprise a ring. The ring may comprise a flexible ring that is configured to rotate about its own axis, with an inner portion 304 of the ring being rotated to then form an outer portion 306 of the ring and continuing to be rotated to return to being an inner portion 304 of the ring, with the cycle continuing for a desired number of rotations. The ring may have sufficient strength to apply a compressive force to the implant.

FIG. 26 illustrates an embodiment of a plurality of rollers 308a-d that may be utilized as a crimping device for an implant. The rollers 308a-d may surround a channel 310 and may be configured to press against the implant and roll as the implant passes through the channel 310. The rolling motion of the rollers 308a-d may provide a mechanical advantage for compressing the implant within the channel 310. The rollers 308a-d shown in FIG. 26 may be coupled to an axle body 311. The rollers 308a-d may be configured to rotate relative to the axle body 311 and may have an outer diameter that is larger than an outer diameter of the axle body 311 to allow the rollers 308a-d to rotate and apply a force to the implant with the axle body 311 remaining non-rotational. The rollers 308a-d and axle body 311 may together form a ring surrounding the channel 310. The rollers 308a-d and axle body 311 may have sufficient strength to apply a compressive force to the implant 82.

The roller 300 shown in FIG. 25 and the plurality of rollers 308a-d shown in FIG. 26 may be slid along an implant 82 to compress the implant 82. As such, a diameter of the channel 302, 310 may be sized to a desired compressed diameter of the implant 82, such that the implant 82 compresses to the diameter of the channel 302, 310.

As shown in FIGS. 25 and 26, the crimping device may comprise at least one roller surrounding a channel 302, 310 configured for the implant to be passed through. The at least one roller is configured to press against the implant to compress the implant and roll as the implant passes through the channel 302, 310. The at least one roller may be rolled over at least a portion of an implant 82 to compress the implant 82. The implant 82 may be passed through a channel 302, 310 surrounded by the at least one roller.

In certain embodiments, other devices may be utilized as part of a crimping device in combination with the rollers 300, 308a-d. FIG. 27 for example illustrates a crimping device 313 including a pull body 312 that is configured to extend over the roller 300 (the pull body 312 may also extend over the rollers 308a-d in another embodiment) and be positioned between the roller 300 and the implant 82. The pull body 312 may be configured to be pulled to roll the roller 300. The pull body 312, for example, may comprise a flexible sheet that is wrapped around the roller 300. The pull body 312 may continue the channel 302 formed by the roller 300 to comprise an implant retention region of the crimping device 313.

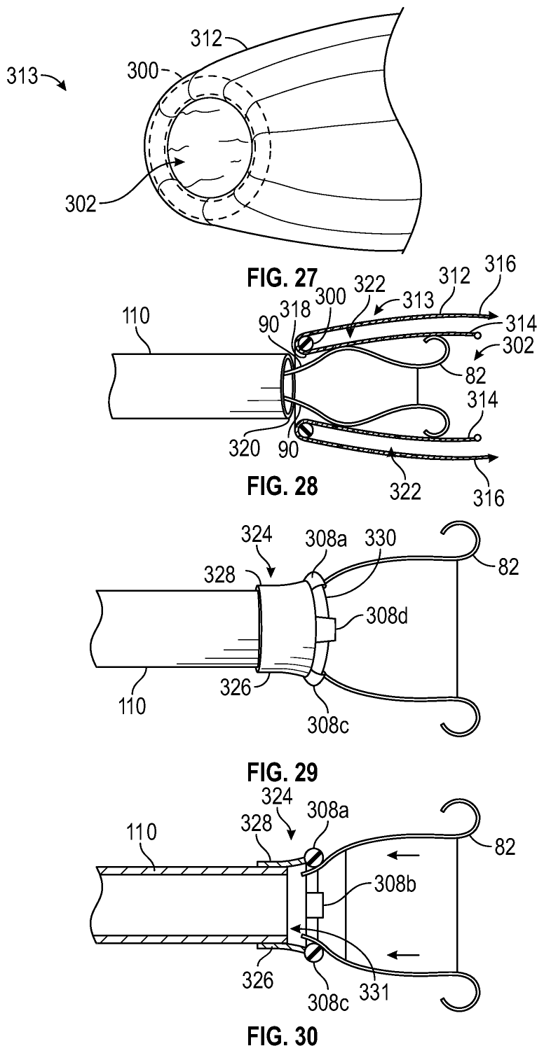

FIG. 28, for example, illustrates a cross sectional view of the crimping device 313 showing an implant 82 positioned within the channel 302. The pull body 312 may be looped around the roller 300 with a first end 314 fixed in position, and a second end 316 configured to be pulled to roll the roller 300. The second end 316 of the pull body 312 may be pulled in a direction away from the roller 300 to cause the roller 300 to rotate. The pull body 312 accordingly may operate as a pulley providing a mechanical advantage for compression of the implant 82. The roller 300 may roll along the body of the implant 82, with the implant 82 being compressed to the inner diameter of the roller 300. The implant 82 may be positioned within the channel 302 such that struts 90 (which may include end tab portions 92) extend from the opening 318 of the crimping device 313. The struts 90 may then be coupled to a coupler of a delivery apparatus, for example, a coupler 94 as shown in FIG. 12. The capsule 110 of the delivery apparatus may be advanced over a portion of the implant 82 after the pull body 312 has been pulled. The remainder of the implant 82 may then be passed through the opening 318 to be inserted into a capsule 110.

In an embodiment as shown in FIG. 28, the capsule 110 may be advanced in a direction towards the implant 82 and over a portion of the implant 82 after the pull body 312 is pulled in a direction away from the capsule 110. The capsule 110 may be advanced as the pull body 312 is being pulled. A distal end 320 of the capsule 110 may be held proximate to the opening 318 of the crimping device 313 to allow the compressed implant 82 to be passed into the capsule 110 as the crimping device 313 is slid away from the capsule 110. In other embodiments, other methods of coupling the implant 82 to a delivery apparatus may be utilized. For example, the crimping device 313 may be disassembled upon the implant 82 being coupled to a coupler of a delivery apparatus, for example, a coupler 94 as shown in FIG. 12. In another embodiment, a portion of a delivery apparatus may be inserted into the channel 302 at the end of the pull body 312 having the fixed end 314, to couple to the implant 82.

In other embodiments, the number of rollers 300 positioned within the pull body 312 may be increased. For example, multiple rollers 300 may be positioned within the gap 322 between the outer and inner portions of the pull body 312. One or more rollers 300 configured as shown in FIG. 25 may be utilized or one or more rollers 308a-d as shown in FIG. 26 may be utilized with the crimping device 313 as desired.

FIG. 29 illustrates an embodiment of a crimping device 324 that includes a support body 326 configured to couple to a portion of a delivery apparatus and configured to couple to one or more of the rollers 300, 308a-d. The rollers 308a-d are illustrated in FIG. 29, however the roller 300 may be utilized with the crimping device 324 as desired in another embodiment.

The support body 326 may include a coupling portion 328 configured to couple to a portion of the delivery apparatus and a coupling portion 330 configured to couple to the rollers 308a-d. The coupling portion 328 for example may comprise a sleeve or other structure for coupling to the delivery apparatus. The support body 326 may form a channel for the implant to be passed through. The coupling portion 328 may be configured to couple to a capsule 110 of the delivery apparatus for retaining the implant, and thus may comprise a sleeve or other structure that extends around an outer surface of the capsule 110 at an end of the capsule 110. The coupling portion 328 may be configured to removably couple from the delivery apparatus such that the crimping device 324 may be removed from the delivery apparatus after the crimping device 324 has been utilized. For example, the coupling portion 328 may be slid off of the capsule 110 after use.

The coupling portion 330 may comprise loops of material or another form of coupler that couples the rollers 308a-d to the support body 326. For example, a loop of material may extend over the axle body 311 shown in FIG. 26, with the outer surfaces of the rollers 308a-d remaining uncovered. The outer surfaces of the rollers 308a-d may thus contact and roll relative to an implant 82 while the non-rotating axle body 311 is coupled to the support body 326. In an embodiment in which the roller is configured as shown in FIG. 25, the coupling portion 330 may comprise a bearing surface (which may loop around a portion of the roller 300) that the roller 300 rotates relative to, with portions of the roller 300 being uncovered to contact the implant 82. For example, portions of the roller 300 that are not covered by the bearing surface may have a larger diameter than portions covered by the bearing surface, such that the uncovered portions of the roller 300 are configured to contact the implant 82.

The support body 326 may comprise a rigid body that is configured to support the rollers 308a-d. The support body 326 may be formed as a funnel, and may hold the rollers 308a-d at a larger diameter portion of the funnel. As such, the rollers 308a-d may serve to compress and reduce the outer diameter of the implant 82 as the implant is inserted through the crimping device 324.

FIG. 30, for example, illustrates a cross sectional view of the crimping device 324 coupled to a capsule 110 of a delivery apparatus. As the implant 82 is inserted through a channel 331 formed by the rollers 308 and that the rollers 308 surround and the support body 326 for the implant 82 to be passed through and into the capsule 110, the rollers 308a-d may roll to compress the implant 82. The channel 331 forms an entry for the capsule 110. The rollers 308a-d may continue to roll upon the implant 82 being fully passed through the channel 331. The implant 82 may be passed in a direction through the channel 331 and into the capsule 110. The crimping device 324 may then be removed from the capsule 110. The configuration of the crimping devices shown in FIGS. 25-30 may be varied in other embodiments.

The embodiments of crimping devices disclosed in regard to FIGS. 25-30 may be utilized solely, or in combination with features of other systems, apparatuses, or methods disclosed herein.

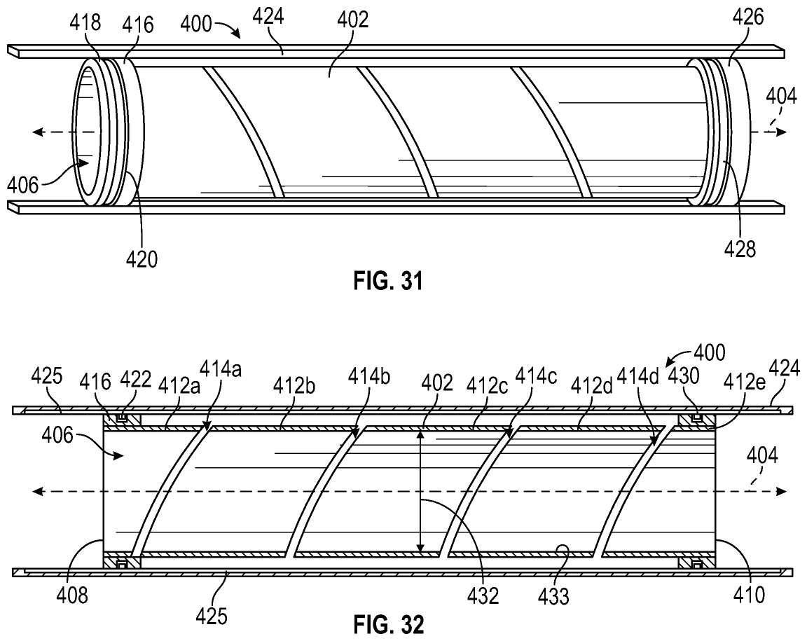

FIG. 31 illustrates a perspective view of an embodiment of a crimping device 400 including a spiral body 402 having an interior diameter 432 (marked in FIG. 32) and extending around an axis 404 and surrounding a channel 406 configured to receive an implant 82. The spiral body 402 is configured to rotate about the axis 404 to reduce the interior diameter 432 and compress the implant 82 within the channel 406.

The spiral body 402 includes ends 408,410 (marked in FIG. 32) and a plurality of wraps 412a-e (marked in FIG. 32) positioned between the ends 408,410. Gaps 414a-d (marked in FIG. 32) may be positioned between the wraps 412a-e in certain embodiments, and in certain embodiments may be excluded (e.g., with the wraps 412a-e touching each other or overlapping each other). The wraps 412a-e may be configured to rotate about the axis 404.

A coupler 416 may be coupled to an end 408 of the spiral body 402 and may comprise a ring extending around the end 408 of the spiral body 402, or may comprise another structure. The coupler 416 may include an outer surface 418 configured to be gripped by a user and may include a channel 420 extending circumferentially around the outer surface 418. The channel 420 may be configured to receive a protrusion 422 (marked in FIG. 32) that is coupled to a housing 424. The channel 420 may be configured for the protrusion 422 to slide within, to allow the coupler 416 to rotate relative to the housing 424. Similarly, a coupler 426 may be coupled to the opposite end 410 of the spiral body 402 and may similarly comprise a ring extending around the end 410. The coupler 426 may similarly include an outer surface having a channel 428 configured to receive a protrusion 430 of the spiral body 402. The couplers 416, 426 (together or separately) may be configured to be rotated to compress the implant 82 within the channel 406.

The housing 424 may comprise a body coupling the ends 408, 410 of the spiral body 402 together. The housing 424 may be configured to prevent the ends 408, 410 of the spiral body 402 from deflecting transverse to the axis 404 upon rotation of the ends 408, 410 about the axis 404. The housing 424 as shown in FIG. 31 may comprise arms extending between the couplers 416, 426, or in other embodiment may have other forms. The housing 424 may be configured to allow the protrusions 422, 430 shown in FIG. 32 to slide longitudinally along the axis 404. As such, the housing 424 may include a track 425 (marked in FIG. 32) that allows the protrusions 422, 430 to slide longitudinally along the axis 404 while the couplers 416, 426 rotate about the axis 404 relative to the protrusions 422, 430. As such, the housing 424 may be configured to accommodate both rotational motion of the couplers 416, 426 about the axis 404 as well as longitudinal motion of the couplers 416, 426 along the axis 404. Thus if a distance between the couplers 416, 426 varies due to rotation of the spiral body 402 then the couplers 416, 426 may be configured to move longitudinally along the axis 404 to account for the varied distance. The spiral body 402 accordingly may have a length that varies as the spiral body 402 rotates about the axis 404. The housing 424 may be configured for the couplers 416, 426 to slide relative to. In other embodiments, the couplers 416, 426 may not be configured to move longitudinally, and may remain fixed in longitudinal position upon rotation of the couplers 416, 426. For example, in such an embodiment the spiral body 402 may be made of a flexible and stretchable material to accommodate a longitudinal force upon the spiral body 402 caused by rotation of the couplers 416, 426.

The spiral body 402 in embodiments may comprise a flexible material, configured to rotate about the axis 404, yet is configured to apply a radial force inward towards the axis 404 to compress the implant 82.

FIG. 32 illustrates a cross sectional view of the crimping device 400. The spiral body 402 has an interior diameter 432. The interior diameter 432 may vary upon relative rotation of the ends 408, 410 of the spiral body 402 in opposite directions about the axis 404 and may decrease to compress an implant 82 positioned within the channel 406. FIG. 34, for example, shows a reduced interior diameter 432 that accordingly compresses the implant 82 within the channel 406. An interior surface 433 of the spiral body 402 is configured to apply a compressive force to the implant 82 to compress the implant 82 within the channel 406.

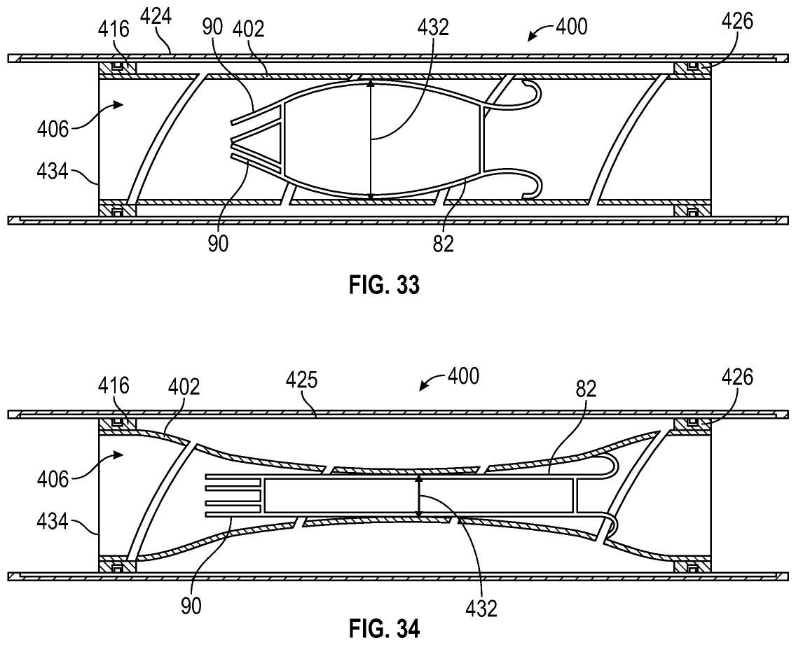

FIG. 33 illustrates cross sectional view of the implant 82 within the channel 406. The spiral body 402 may be in an unrotated state, having a relatively large interior diameter 432. The implant 82 may be positioned within the channel 406 with the longitudinal axis of the implant 82 extending along the longitudinal axis 404 (marked in FIG. 32) of the spiral body 402. Struts 90 of the implant 82 that may end in end tab portions 92 may extend towards an opening 434 an at end of the spiral body 402 for the implant to be passed through.

FIG. 34 illustrates a cross sectional view of the crimping device 400 in which the spiral body 402 has been rotated to reduce the size of the interior diameter 432 and thus compress the implant 82. The spiral body 402 may be rotated by one or more of the couplers 416, 426 being gripped and rotated in directions relative to each other that are opposite about the axis 404. A single one of the couplers 416, 426 may be rotated relative to the other of the couplers 426, 416, or both couplers 416, 426 may be rotated in directions that are opposite to each other about the axis 404. The reduced size of the interior diameter 432 may vary the distance between the couplers 416, 426 longitudinally, which may be accommodated by the use of the track 425.

The implant 82 may be compressed such that the struts 90 are positioned for coupling to a coupler of a delivery apparatus, for example, a coupler 94 as shown in FIG. 12. The coupler 94 may be inserted into the channel 406 to couple to the struts 90 and may couple to end tab portions 92 of the struts 90 while the implant is compressed within the channel 406. In other embodiments, the struts 90 may protrude from the opening 434 for coupling to a coupler of a delivery apparatus, for example, a coupler 94 as shown in FIG. 12. Thus, the coupler may not need to be inserted into the channel 406. Upon coupling to a coupler while the implant 82 is compressed within the channel 406, the implant 82 may be slid out of the channel 406 and into a capsule 110 of a delivery apparatus. In embodiments, upon coupling to a coupler, the spiral body 402 may be unwrapped progressively along the length of the implant 82 as the implant 82 is inserted into a capsule 110 of a delivery apparatus. At least a portion of the spiral body 402 may be configured to be unwrapped from the implant 82, In embodiments, the spiral body 402 may be separated or otherwise disassembled from other components of the crimping device 400 and may be inserted into the capsule 110 along with the implant 82 compressed within the spiral body 402. In such an embodiment, the spiral body 402 may remain a sheath extending around the implant 82 that is inserted into the capsule 110 along with the implant 82.

The configuration of the crimping device 400 may be varied in other embodiments.

The embodiments of crimping devices disclosed in regard to FIGS. 31-34 may be utilized solely, or in combination with features of other systems, apparatuses, or methods disclosed herein.

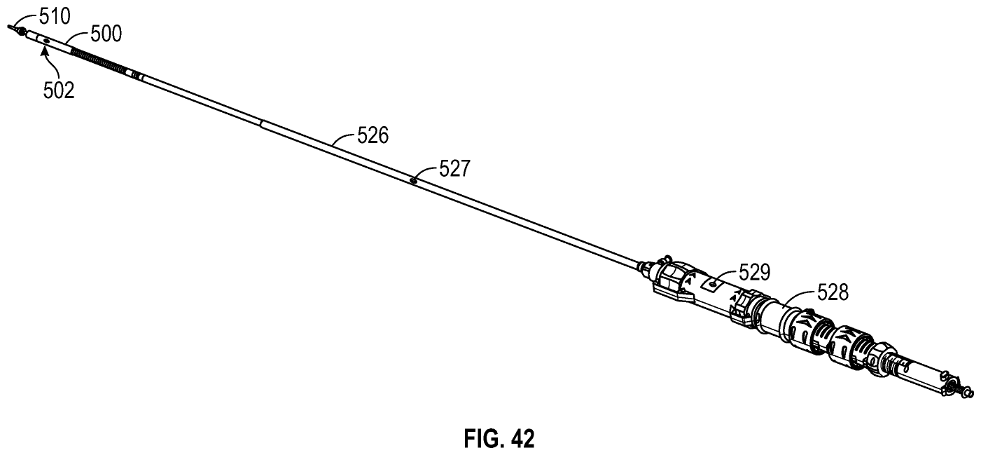

The crimping devices disclosed herein may be utilized for loading an implant into a delivery apparatus. The delivery apparatus may have a variety of forms, one such exemplary form being shown in FIG. 42. The delivery apparatus may include a handle 528 and an elongate shaft 526 having a proximal end coupled to the handle 528 and a distal end including a capsule 500 surrounding an implant retention area. The delivery apparatus may include actuation mechanisms (as shown on the handle 528 in FIG. 42) that may be utilized to operate features of the delivery apparatus, including deployment of the implant (such as via retraction of the capsule), or other features which may include flexing of the elongate shaft 526 to position the elongate shaft 526 in a desired orientation. The delivery apparatuses may be utilized in delivery systems for deploying an implant to a desired location within the human body. The delivery apparatuses may comprise delivery catheters configured to be inserted through a lumen of a human body to reach a desired deployment location for the implant. Various other configurations of delivery apparatuses may be utilized.

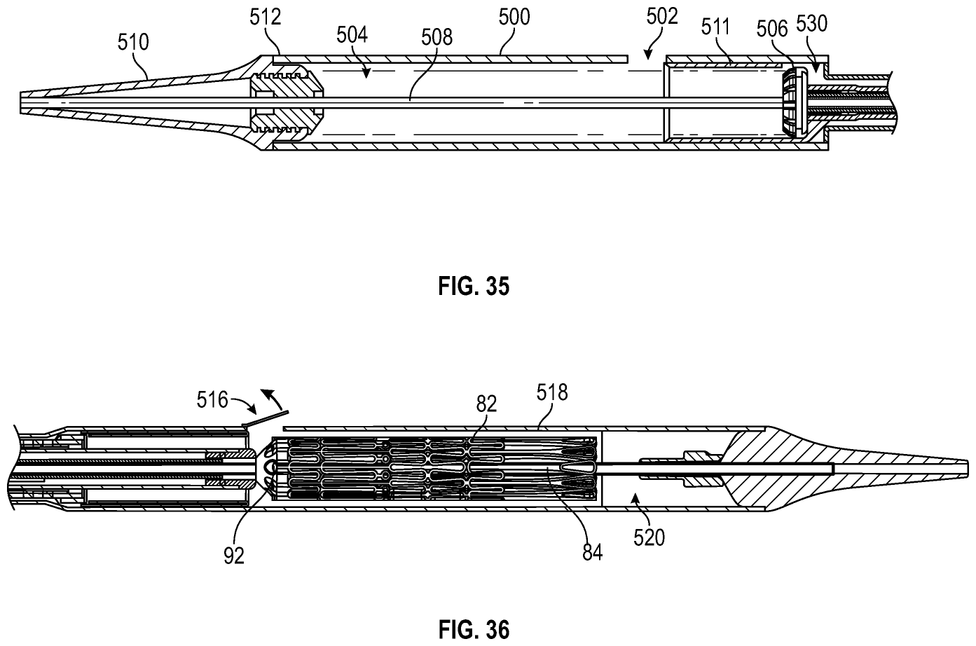

The delivery apparatus may be configured to accommodate for insertion or deployment of the implant 82 from an implant retention area. FIG. 35, for example, illustrates an embodiment of a delivery apparatus including a capsule 500 having a port 502. The capsule 500 may comprise a sheath surrounding an implant retention area 504 for retaining an implant 82. The delivery apparatus may include a coupler 506 configured to retain the implant 82 that operates similarly as the coupler 94 shown in FIG. 12. An elongate shaft 508 may extend within the implant retention area 504 to couple to a nose cone 510 at a distal end of the delivery apparatus.

The delivery apparatus may further comprise a retaining ring 511 configured to extend around a proximal end of the implant 82. The retaining ring 511 may be positioned within the capsule 500.

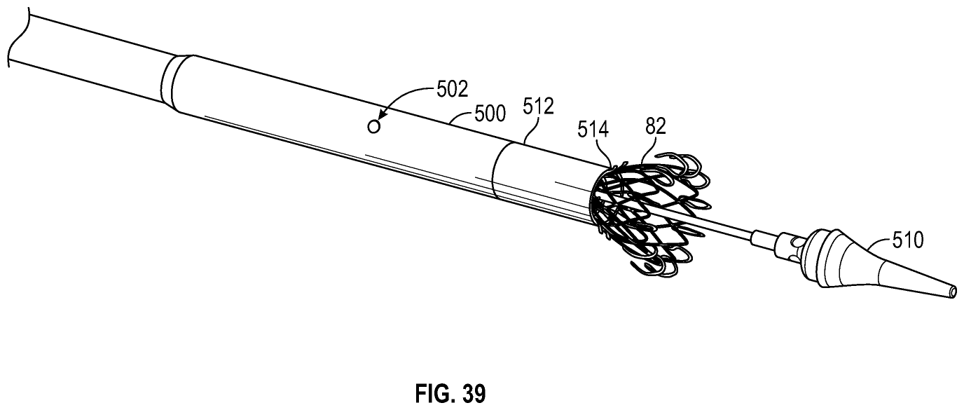

The capsule 500 may have a distal end 512 that forms an opening 514 (marked in FIG. 39) for the implant 82 to be deployed from at a desired location within a patient's body. The opening 514 may also be configured for the implant 82 to be inserted through to be positioned within the implant retention area 504. Referring first to the insertion of the implant 82 within the implant retention area 504, the implant 82 may be inserted through the opening 514 in a proximal direction along the elongate shaft 508. The nose cone 510 may be displaced, similar to the configuration of the nose cone 112 shown in FIG. 12. A proximal end of the implant 82 may be coupled to the coupler 506 as discussed herein, and the implant 82 may continue to be slid into the implant retention area 504 through the opening 514. A crimping device, such as a crimping device disclosed herein may be utilized to assist in the insertion of the implant 82 into the implant retention area 504.

As the implant 82 is inserted proximally through the opening 514 into the implant retention area 504, a fluid pressure may build within the implant retention area 504. The fluid may comprise a gas (such as air or another gas) or a liquid if applicable. The fluid pressure within the implant retention area 504 may impede the ability of the implant 82 to be inserted into the implant retention area 504 or may otherwise provide a resistive force to the insertion of the implant 82.

The port 502 may be configured to allow for fluid transfer into or out of the capsule 500. With the port 502 able to allow for fluid transfer out of the capsule 500 upon the implant 82 being inserted into the capsule 500 through the opening 514, the fluid pressure within the capsule 500 may be relieved, reducing the resistive force to the insertion of the implant 82. The port 502 may release fluid out of the capsule 500 that is pressed by the implant 82 upon the implant 82 being inserted into the capsule 500 through the opening 514. In embodiments, the port 502 may be configured to allow for fluid transfer into and out of the capsule 500.

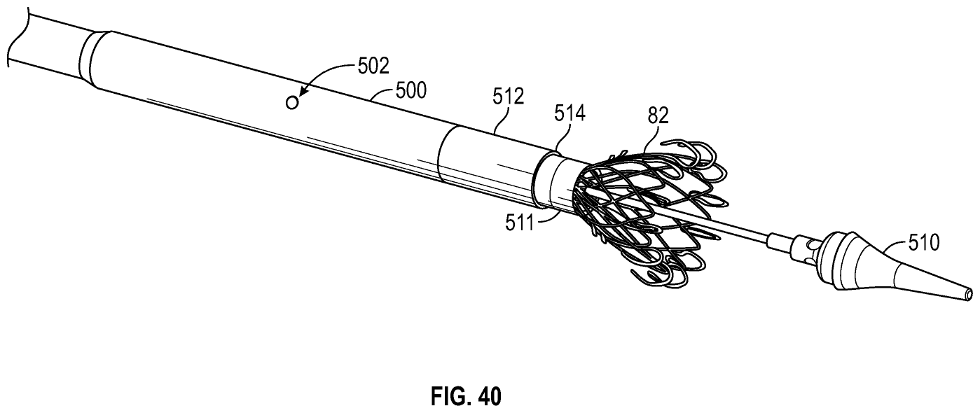

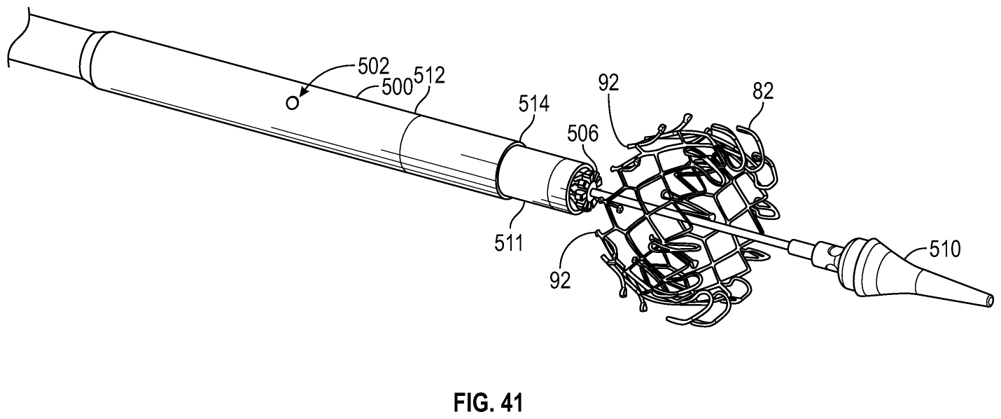

With regard to the deployment of the implant 82 from the implant retention area 504, the implant 82 may be passed back through the opening 514 in a distal direction. FIGS. 39-41 for example illustrate a process of releasing the implant 82 from the implant retention area 504 by the capsule 500 being retracted proximally relative to the nose cone 510 and the implant 82. FIG. 39 for example illustrates the capsule 500 initially being retracted with a distal portion of the implant 82 being exposed and expanded from the capsule 500. FIG. 40 illustrates the capsule 500 being fully retracted with the retaining ring 511 remaining positioned over the proximal end of the implant 82. FIG. 41 illustrates the retaining ring 511 retracted to allow the implant 82 to release from the delivery apparatus, by uncovering the end tab portions 92 that were coupled to the coupler 506. In such a process, a pressure gradient from within the capsule 500 relative to outside the capsule 500 may impede the ability of the capsule 500 to be retracted or the implant 82 being released from within the capsule 500. Such a pressure gradient may be a vacuum force from within the capsule 500. The port 502, accordingly, may be configured allow for fluid transfer into the capsule 500 upon the implant 82 being released from the capsule 500 through the opening 514 to relieve the pressure gradient and reduce the resistive force caused by the pressure gradient during deployment of the implant 82. The port 502 may pass fluid into the capsule 500 through the port 502 upon the implant 82 being released from the capsule 500.

The port 502 may have a variety of forms, and referring to FIG. 35 may comprise an opening extending through the capsule 500 and particularly an outer surface of the capsule 500. The opening may allow fluid to transfer out of the capsule 500 upon insertion of the implant 82 into the implant retention area 504 and may allow fluid to transfer into the capsule 500 upon release of the implant 82 from the implant retention area 504. The port 502 may be positioned on a proximal portion of the capsule 500 such that a pressure gradient is equalized at a portion of the implant retention area 504 covering a proximal portion of the implant 82. As such, as the implant 82 is pressed into the capsule 500, the pressure caused by the proximal end of the implant 82 is relieved, and as the implant 82 is released from the capsule 500, the pressure gradient at the proximal end of the implant 82 is relieved as well. Further, as the implant 82 is released from the capsule 500, the port 502 may pass fluid into the capsule 500 in a region of the capsule 500 that the implant 82 is released from. In certain embodiments the retaining ring 511 may include a port that allows fluid transfer into or out of the interior of the retaining ring 511 as well.

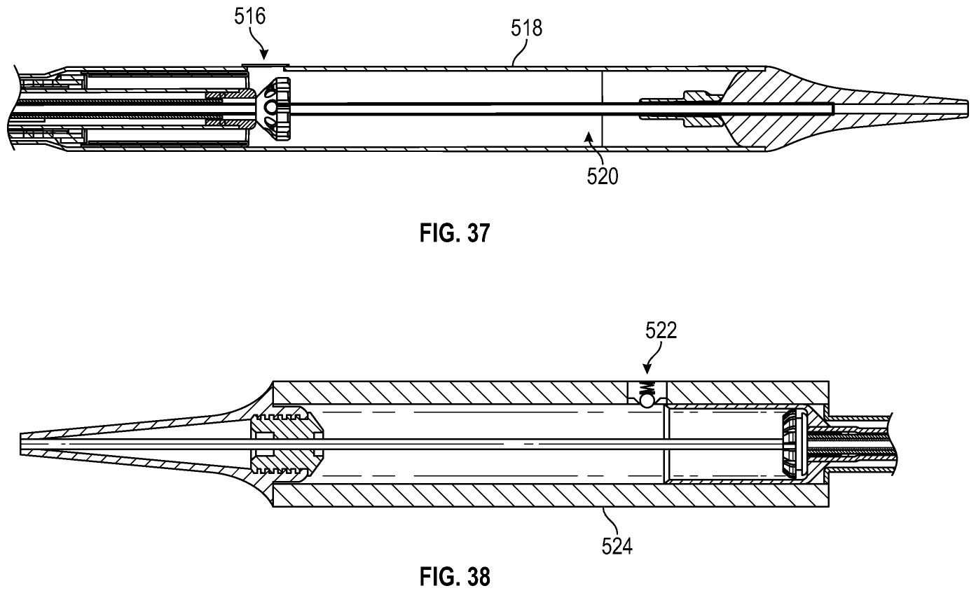

In embodiments, the port 502 may comprise a valve, which may have a variety of forms. A valve for example may comprise a check valve that may only allow for fluid transfer in a particular direction into or out of the capsule. FIG. 36 for example, illustrates a port 516 in the form of a check valve that only allows fluid transfer out of the capsule 518. Such a feature may allow the port 516 to relive pressure upon insertion of the implant 82 into the implant retention area 520, but may not allow the port 516 to relieve pressure upon the implant 82 released from the implant retention area 520. The closed check valve may prevent air or other undesired substances from passing through the implant retention area 520 upon release of the implant 82 from the implant retention area 520 or upon insertion or removal of the delivery apparatus from a portion of the patient's body. FIG. 37, for example, illustrates the port 516 may remain closed after release of the implant 82 from the implant retention area 520.

In other embodiments, the port may comprise a check valve configured to only allow fluid transfer into the capsule 518.

FIG. 38 illustrates a port 522 in the form of a pressure release valve. The pressure release valve may have a threshold pressure gradient that must be met to cause the port 522 to open. In such a configuration, the port 522 may only operate to allow fluid transfer into or out of the capsule 524 upon a threshold gradient being met. The port 522 as shown in FIG. 38 is configured to only allow fluid to transfer out of the capsule 524, but the port 522 may be configured to only allow fluid to transfer into the capsule 524 or may allow for fluid transfer into and out of the capsule 524 in other embodiments.

The port may be positioned at one or more locations upon the delivery apparatus. As shown in FIG. 35-41, the ports disclosed herein may be positioned on the capsules of the delivery apparatuses. Referring to FIG. 42, one or more of the ports disclosed herein may be positioned on the capsule 500, on a portion of an elongate shaft 526 extending proximally from the capsule 500 (shown as port 527), and may be positioned on a handle 528 coupled to a proximal end of the elongate shaft 526 (shown as port 529), or may be positioned elsewhere as desired. For the ports 527, 529 on the elongate shaft 526 and the handle 528, a channel 530 (as marked in FIG. 35) may be in fluid communication with the implant retention area 504, to allow for fluid transfer into or out of the capsule 500 as desired. The channel 530 may extend proximally to a portion of the elongate shaft 526 or handle 528 where the ports 527, 529 are located. The channel 530 may be positioned at a proximal end of the capsule 500 or another portion of the elongate shaft 526 as desired.

The embodiments of ports disclosed in regard to FIGS. 35-42 may be utilized solely, or in combination with features of other systems, apparatuses, or methods disclosed herein.

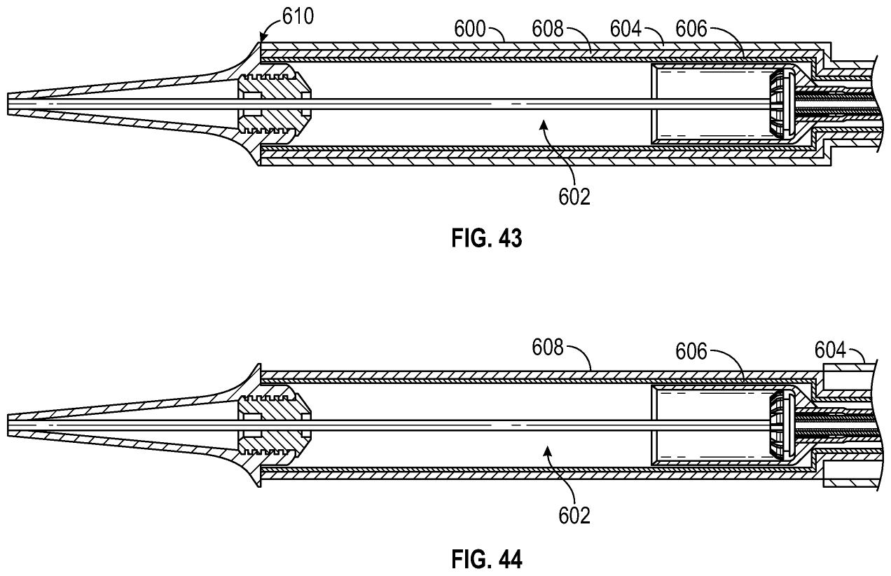

FIG. 43 illustrates an embodiment of a delivery apparatus including a capsule 600 surrounding an implant retention area 602 for retaining an implant 82. The capsule 600 is a multi-layer capsule and includes a plurality of layers each configured to be retracted separately to deploy the implant 82 from the implant retention area 602. The capsule 600 as shown in FIG. 43 may include an outer layer 604 extending around an inner layer 606. An intermediate layer 608 is shown in FIG. 43 and extends around the inner layer 606 and is sandwiched between the outer layer 604 and the inner layer 606. The outer layer 604 extends around the intermediate layer 608. The layers 604, 606, 608 may be configured to slide relative to each other (e.g., the outer layer 604 may be configured to slide relative to the inner layer 606 and the intermediate layer 608, the intermediate layer 608 may be configured to slide relative to the inner layer 606). In other embodiments, the intermediate layer 608 may be excluded. In other embodiments, the number of layers may be increased from three layers as shown in FIG. 43 (e.g., 4 layers, 5 layers).

The layers of the capsule 600 may be configured to be retracted separately such that the deployment force of an implant 82 retained with the implant retention area 602 is gradually reduced through the retraction of the separate layers. Thus, the multi-layered configuration may provide a strong capsule for delivery to a portion of the patient's body and retention of the compressed implant, yet may have the thickness of the capsule gradually reduced during deployment of the implant. In such a manner, the force required to retract the capsule 600 may be reduced based on the force required to retract the individual layers (rather than the capsule 600 as a whole). Further, the friction force applied by the capsule 600 to the implant as the capsule 600 is retracted may be reduced due to the reduced rigidity of the capsule 600 caused by the reduced thickness of the capsule at the time of implant release.

A hardness of each of the layers of the capsule 600 may be different than each other, with an outermost layer having a highest hardness and an innermost layer having a lesser hardness. As such, the friction force applied by the inner most layer 606 of the capsule 600 to the implant as the inner most layer 606 is retracted may be reduced from the friction force that would be provided by the entire capsule 600 being retracted at once. Further, the outermost layer may have the highest hardness to protect the implant from force applied to the outside surface of the capsule 600. In other embodiments, each of the layers of the capsule 600 may have the same hardness.

The layers of the capsule 600 may each be coupled to separate actuation mechanisms for separately retracting each of the plurality of layers. Thus, a user may be able to control which of the layers is retracted and in what order, and may be able to control the amount of retraction of each of the layers. The plurality of layers may each comprise a sheath extending proximally from the implant retention area 602 to a handle of the delivery apparatus. Each sheath accordingly may be retracted by the actuation mechanism of the handle separately. The plurality of layers may be configured to be retracted sequentially, starting with an outermost one of the plurality of layers. In embodiments, the layers of the capsule 600 may be retracted separately by the layers being coupled to each other in a manner that causes a delayed retraction of one layer in response to retraction of another layer. For example, the intermediate layer may be coupled to the outermost layer such that as the outermost layer is retracted a certain distance then retraction of the intermediate layer will start. Thus, a user would only need to retract the outermost layer to also automatically retract the intermediate layer and then the inner layer (as the inner layer may be coupled to the intermediate layer in a similar manner).

FIG. 43 illustrates the layers 604, 608, 606 of the capsule 600 in position around the implant retention area 602. A distal end of each of the layers is positioned at a distal end of the capsule 600 including an opening 610 of the capsule 600 for the implant to be inserted into or deployed from (similar to opening 514 shown in FIG. 39). A distal end of each of the layers is positioned at the opening 610.

FIG. 44 illustrates that the outer layer 604 has been retracted relative to the inner layer 606 and intermediate layer 608, leaving the intermediate layer 608 and inner layer 606 extending around the implant retention area 602.

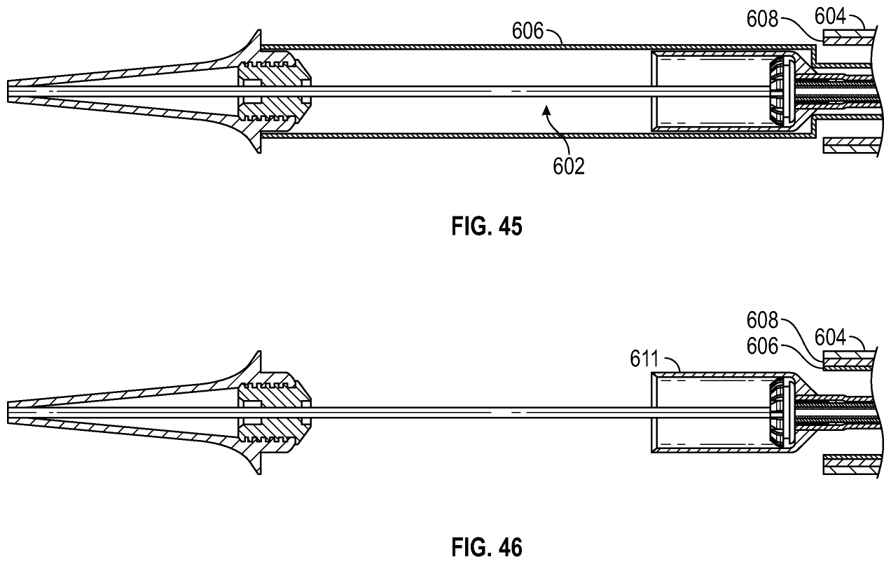

FIG. 45 illustrates that the intermediate layer 608 has been retracted, leaving the inner layer 606 extending around the implant retention area 602. The frictional force applied by the inner layer 606 to the implant 82 upon retraction of the inner layer 606 is reduced from the frictional force that would be applied by the entire capsule 600 to the implant 82. Further, the force required to retract the inner layer 606 is less than the force required to retract the entire capsule 600.

FIG. 46 illustrates that the inner layer 606 has been retracted to uncover the implant retention area 602 and deploy the implant from the capsule 600. The inner layer 606 has been retracted to expose at least a portion of the implant 82. A retaining ring 611 may be released to fully release the implant from the delivery apparatus.

In other embodiments, the sequence of retraction of the layers may be varied from the sequence shown in FIGS. 43-46, for example, an intermediate layer or inner layer may first be retracted followed by an outer layer if desired. Other sequences may be utilized as desired. The sequence shown in FIGS. 43-46 includes separately retracting a plurality of layers 604, 606, 608 of a multi-layer capsule 600 extending over an implant retention area 602 to deploy an implant from the implant retention area 602. The plurality of layers 604, 606, 608 are sequentially retracted starting with the outermost one of the layers 604. The outer layer is retracted relative to the intermediate layer and the intermediate layer is retracted relative to the inner layer. An actuation mechanism may be operated on the handle to separately retract the plurality of layers 604, 606, 608, among other methods of retraction.

The embodiments of capsules disclosed in regard to FIGS. 43-46 may be utilized solely, or in combination with features of other systems, apparatuses, or methods disclosed herein.

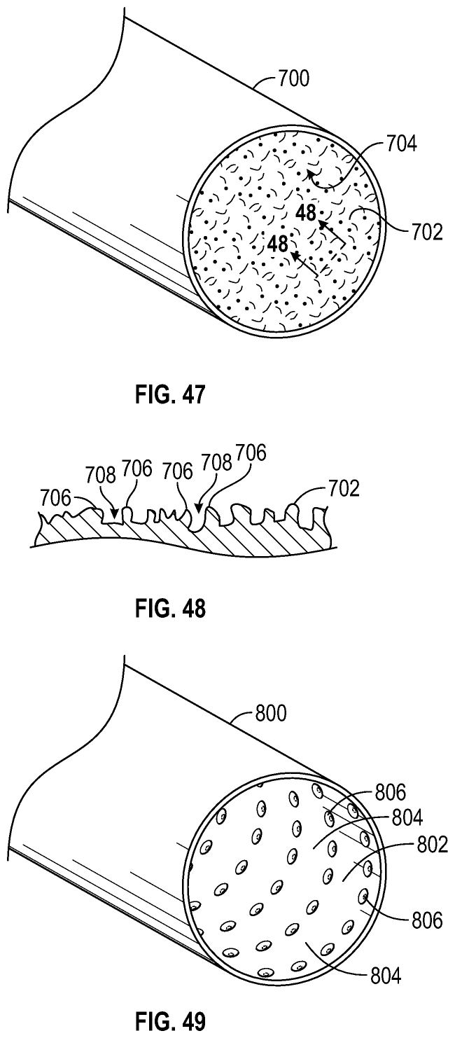

FIG. 47 illustrates a perspective view of a capsule 700 of a delivery apparatus having an inner surface 702 that is configured to face towards an implant 82 positioned within an implant retention area 704 that the capsule 700 surrounds. The inner surface 702 may include a plurality of protrusions 706 (marked in FIG. 48) for the implant 82 to slide along. FIG. 48 illustrates a cross-sectional close-up view of the inner surface 702 showing the plurality of protrusions 706. The plurality of protrusions 706 may be formed by a textured material that may form the inner surface 702 of the capsule 700. The textured material may comprise a micro textured material that may have a roughened profile or irregular profile.

FIG. 48, for example, illustrates the protrusions 706 comprising raised portions of the inner surface 702 and being separated by indentations 708. The protrusions 706 may be configured for the implant 82 to slide along to reduce frictional force from the frictional force that would be provided if the inner surface 702 were smooth. As such, the tips of the protrusions 706 may contact the implant 82, providing a lesser contact surface area against the implant 82 than if the entire surface area of the inner surface 702 contacted the implant 82.

Such a feature may reduce the insertion force of the implant 82 being inserted into the capsule 700 and may reduce the deployment force of the implant 82 being deployed from the capsule 700.

FIG. 49 illustrates a perspective view of a variation of the capsule 700, in which the inner surface 802 of a capsule 800 includes a plurality of protrusions in the form of raised portions 804 of the inner surface 802 separated by indentations 806. The indentations 806, for example, may comprise divots in the inner surface 802. The presence of the indentations 806 may reduce the contact surface area of the inner surface 802 upon the implant as the capsule 800 is slid relative to the implant 82 (during either insertion into the capsule 800 or deployment from the capsule 800).

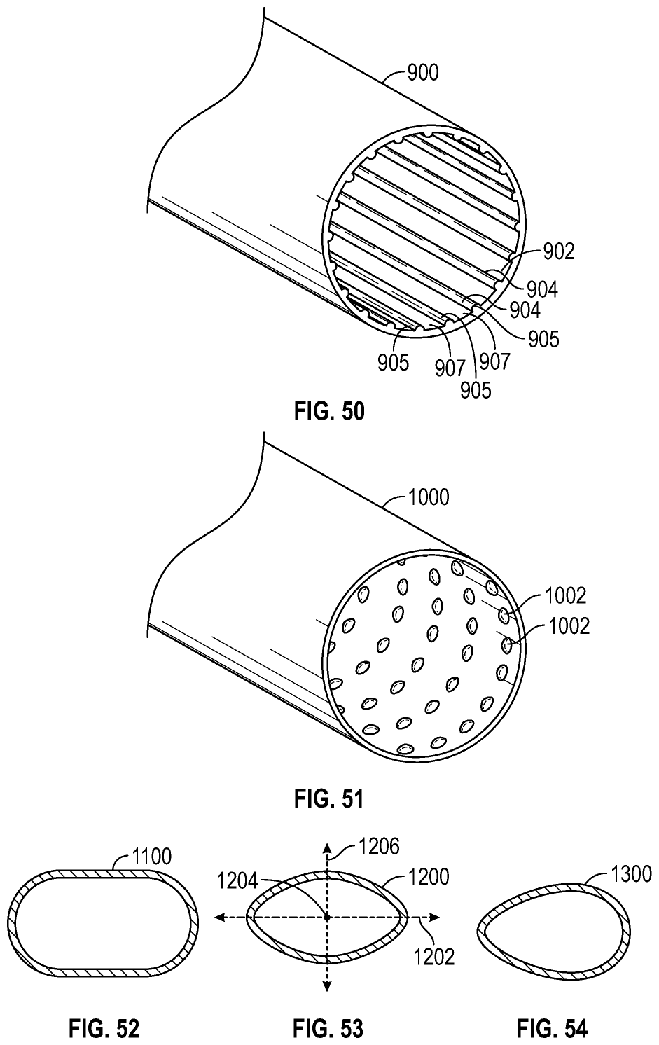

FIG. 50 illustrates a perspective view of a variation of the capsule 700, in which the inner surface 902 of the capsule 900 includes a plurality of protrusions in the form of bumps 904 extending longitudinally along the capsule 900. The bumps 904 may include contact portions 905 for contacting the implant and may be separated from each other by channels 907. The bumps 904 may comprise ribs extending longitudinally parallel with the longitudinal axis that the capsule 900 surrounds (as shown in FIG. 50) or may comprise ribs extend longitudinally offset from the axis that the capsule 900 surrounds. The ribs may be equally spaced about the longitudinal axis of the capsule 900. The raised contact portions 905 of the ribs may reduce the contact surface area of the inner surface 902 upon the implant as the capsule 900 is slid relative to the implant 82 (during either insertion into the capsule 900 or deployment from the capsule 900).

FIG. 51 illustrates a perspective view of a variation of the capsule 700, in which the inner surface of a capsule 1000 includes a plurality of protrusions in the form of bumps 1002. The bumps 1002, similar to the bumps shown in FIG. 50, may include contact portions (the uppermost portion of the respective bump) for contacting the implant. The bumps 1002 may have a circular shape or another shape as desired. The bumps 1002 may be equally spaced from each other or may have another position as desired. The raised contact portions of the bumps 1002 may reduce the contact surface area of the inner surface upon the implant as the capsule 1000 is slid relative to the implant 82 (during either insertion into the capsule 1000 or deployment from the capsule 1000).

The capsules 700, 800, 900, 1000 disclosed herein may be configured to surround an implant retention area for retaining an implant. The respective inner surfaces may be configured to face towards the implant within the implant retention area and may include the plurality of protrusions for the implant to slide along. The implant may be slid relative to the respective capsule 700, 800, 900, 1000 to be deployed from the capsule (e.g., by retracting the respective capsule) or to be inserted into the capsule 700, 800, 900, 1000.

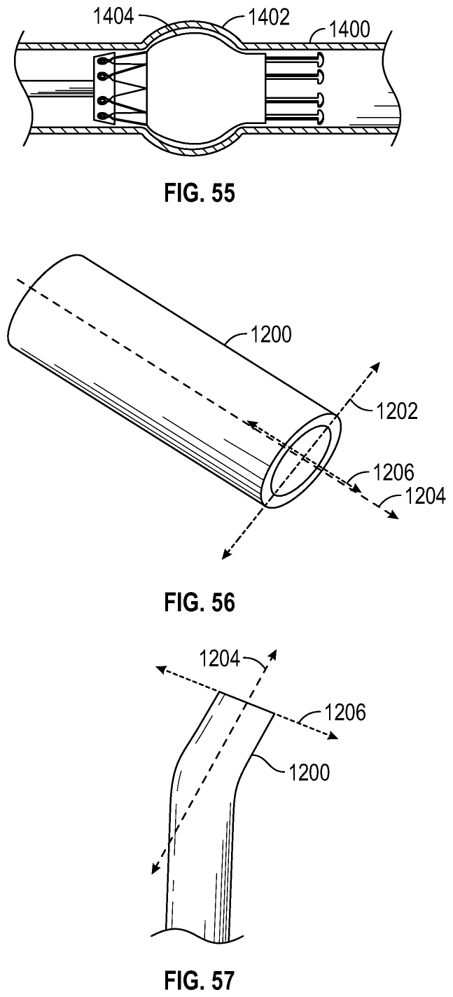

The capsules of the delivery apparatuses disclosed herein may have outer surfaces with uniformly circular cross sectional shapes along a plane perpendicular to the longitudinal axis of the capsules. However, in embodiments, the outer surfaces may have non-uniformly circular cross sectional shapes along the length of the capsule, with the length of the capsule extending along the longitudinal axis of the capsule. FIG. 52, for example, illustrates a cross sectional view of a capsule 1100 along a plane perpendicular to the longitudinal axis of the capsule. The capsule 1100 including an outer surface that is oblong in shape and thus has an oblong cross-sectional shape. FIG. 53, for example, illustrates a cross sectional view of a capsule 1200 along a plane perpendicular to the longitudinal axis 1204 of the capsule that is oval in shape and thus has an oval cross-sectional shape. FIG. 54, for example, illustrates a cross sectional view of a capsule 1300 along a plane perpendicular to the longitudinal axis of the capsule that is ovoid in shape and thus has an ovoid cross-sectional shape, which is a non-circular cross-sectional shape. FIG. 55, for example, illustrates a side cross sectional view of an embodiment of a capsule 1400 that has a non-uniformly circular cross sectional shape along the length of the capsule 1400 because of the presence of a bump portion 1402 along the length of the capsule 1400 having a greater diameter than an adjacent portion of the outer surface (thus varying the diameter of the circular cross sectional shape along the length of the capsule and rendering it non-uniform). The bump portion 1402 may be configured to contour to the shape of the implant. The outer surface of the capsule 1400 has a circular cross-sectional shape that is non-uniform, as it has various diameters along the length of the capsule 1400.

The outer surfaces and inner surfaces of the capsules disclosed herein may have a non-uniformly circular cross sectional shape along the length of the capsule. The inner surface may face opposite the outer surface and may be configured for the implant to slide along. The cross-sectional shape of the inner surface may match the cross-sectional shape of the outer surface in certain embodiments, or may be different in other embodiments.

The non-uniformly circular cross-sectional shapes shown in FIGS. 52-55 may provide a variety of benefits. For example, referring to FIG. 56, which is a perspective view of the capsule 1200 shown in FIG. 53, the capsule 1200 is shown to include a major axis 1202 and a minor axis 1206 (extending in the same plane yet perpendicular to the major axis 1202), and a longitudinal axis 1204 that the capsule 1200 surrounds and extends along. The capsule 1200 thus has a flattened profile. The capsule 1200 is configured to flex in a direction transverse to the length of the capsule 1200. Referring to FIG. 57, if the capsule 1200 is flexed in the plane that the minor axis 1206 extends in, then the deflection force of the capsule 1200 will be reduced because the width of the capsule 1200 in the plane of the minor axis 1206 is less than the width of the capsule 1200 in the plane of the major axis 1202. As such, a thinner capsule 1200 in the dimension of deflection may be easier to deflect than a capsule that is uniformly circular and may have a greater thickness in that desired plane of deflection. Further, the relative position of the major axis 1202 and minor axis 1206 may be set to reduce the deflection force in a desired plane of deflection of the capsule 1200. For example, if it is known that the capsule 1200 will be required to flex in a certain direction at a certain point in a procedure, then the minor axis 1206 may be positioned to be aligned with that direction. Thus, the capsule 1200 may have a reduced bend force in that direction during the procedure.

A benefit of the non-uniformly circular cross sectional shapes shown in FIGS. 52-55 may include allowing the capsules to contour to a shape of the implant retained within the respective capsule (e.g., the implant retention area). For example, FIG. 55 illustrates that the implant 1404 has a large bump section, and the capsule 1400 may be shaped with the bump portion 1402 to allow the capsule 1400 to closely contour to the implant 1404 and reduce unnecessary empty space within the capsule 1400 that would be present if the capsule 1400 had a uniformly circular cross sectional shape along its length. Further, in the embodiments shown in FIGS. 52-54, the implant may have an oblong, or oval, elliptical, or ovoid shape that the respective capsules 1100, 1200, 1300 may contour to. Thus, unnecessary empty space within the capsules 1100, 1200, 1300 may be reduced.

Further, in certain embodiments the wall forming the capsules 1100, 1200, 1300, 1400 and surrounding the implant retention area may be made flexible (i.e., a flexible wall), to allow the respective capsules 1100, 1200, 1300, 1400 to contour to the shape of the respective implant retained within the implant retention area. The capsules 1100, 1200, 1300, 1400 may have an outer surface profile that contours to the shape of the implant retained within the implant retention area. The capsule may be configured to contour as the implant is inserted into the capsules 1100, 1200, 1300, 1400. In such a configuration, the wall forming the capsules 1100, 1200, 1300, 1400 may be configured to deform to contour to the shape of the respective implant retained within the implant retention area. For example, as shown in FIG. 55, the wall forming the capsule 1400 may be configured to deform to provide the bump portion 1402 in the capsule as the implant is inserted into the capsule 1400. The wall may further be configured to stretch to allow the wall forming the capsule 1400 to contour to the shape of the implant. Further, the wall may be resilient to allow the wall to compress to the implant. Such a configuration may reduce unnecessary empty space within the capsule by allowing the respective capsule to contour to the shape of the implant. In embodiments, the capsules 1100, 1200, 1300, 1400 may have a preformed shape that contours to the shape of the implant. For example, the wall forming the capsules 1100, 1200, 1300, 1400 may not be flexible, but may be preformed into a desired shape that contours to the shape of an implant.

The embodiments of capsules disclosed in regard to FIGS. 47-57 may be utilized solely, or in combination with features of other systems, apparatuses, or methods disclosed herein.

An implant may be inserted into or deployed from any of the capsules disclosed herein. Any crimping device as disclosed herein may be utilized with any of the capsules or other features of the delivery apparatuses disclosed herein.

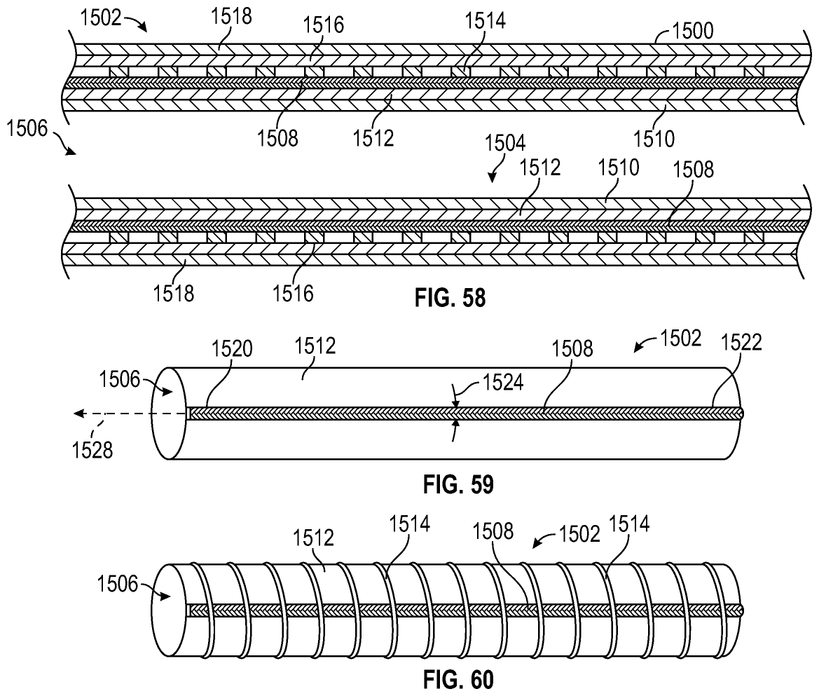

FIG. 58 illustrates a side cross sectional view of a capsule 1500 of an elongate shaft 1502. The capsule 1500 may be configured to surround an implant retention area 1504 that may be configured to retain an implant as disclosed herein. The implant retention area 1504, for example, may comprise a channel 1506 for the implant to be deployed from.

The elongate shaft 1502 may include an elongate flat braid ribbon 1508 that extends along the elongate shaft 1502. The elongate flat braid ribbon 1508 may extend along the portion of the elongate shaft 1502 comprising the capsule 1500 in embodiments. In embodiments, the elongate shaft braid ribbon 1508 may extend over other portions of the elongate shaft 1502.

The elongate shaft 1502 and capsule 1500 may comprise a multi-layered structure in embodiments. As shown in FIG. 58, for example, the elongate shaft 1502 may include an inner liner layer 1510. The liner layer 1510 may be positioned interior of the elongate flat braid ribbon 1508. The inner liner layer 1510 in embodiments may comprise a portion of the elongate shaft 1502 that contacts an implant positioned within the implant retention area 1504. The elongate shaft 1502, for example, may include an outer sheath that extends over the implant retention area 1504, and the inner liner layer 1510 may comprise the inner layer of that outer sheath. The outer sheath may comprise the capsule 1500.

A hypotube 1512, or metal layer or stent, may be positioned radially outward of the inner liner layer 1510. The hypotube 1512 may surround the inner liner layer 1510. In embodiments, the hypotube 1512 may include a cut pattern that allows for flexibility of the hypotube 1512 or another structure that allows for flexibility of the hypotube 1512. In embodiments, the hypotube 1512 may provide structural support for the elongate shaft 1502 and may be configured to transmit an axial force in a distal direction, for example during recapture of an implant from the implant retention area 1504.

The elongate flat braid ribbon 1508 may extend over and along the hypotube 1512 and may be positioned radially outward of the hypotube 1512. The hypotube 1512 may be positioned interior of the elongate flat braid ribbon 1508. In embodiments, a coil layer including a coil 1514 may extend over and along the elongate flat braid ribbon 1508 and may be positioned radially outward of the elongate flat braid ribbon 1508. The coil 1514 may be positioned exterior of the elongate flat braid ribbon 1508. The coil 1514 may wrap over the elongate flat braid ribbon 1508 and may be configured to retain the elongate flat braid ribbon 1508 to the hypotube 1512.

In embodiments, one or more outer layers may be positioned over the coil 1514. The outer layer may be positioned exterior of the coil 1514. For example, as shown in FIG. 58, a first outer layer 1516 or tie layer may be positioned over the coil 1514 and may comprise a material such as expanded polytetrafluoroethylene (ePTFE) or another material. A second outer layer 1518 or outer coating may be positioned over the first outer layer 1516 and may comprise a material such as polyether block amide (PEBAX®) or another material. The second outer layer 1518 may comprise the outer surface of the elongate shaft 1502, which may include the outer surface of the capsule 1500. In embodiments, other materials may be utilized for the layers, and the configuration of the layers may be varied as desired. For example, in one embodiment, the first outer layer 1516 may be excluded.

FIG. 59 illustrates a side perspective view of the elongate flat braid ribbon 1508 extending along the hypotube 1512. The elongate flat braid ribbon 1508 extends longitudinally, from a proximal portion 1520 of the elongate flat braid ribbon 1508 to a distal portion 1522 of the elongate flat braid ribbon 1508. The distal portion 1522 of the elongate flat braid ribbon 1508 in embodiments, may be positioned at the distal end of the capsule 1500, or may have another position as desired. In embodiments, the elongate flat braid ribbon 1508 may be configured to extend for the entire length of the elongate shaft 1502, or a portion of the elongate shaft 1502.

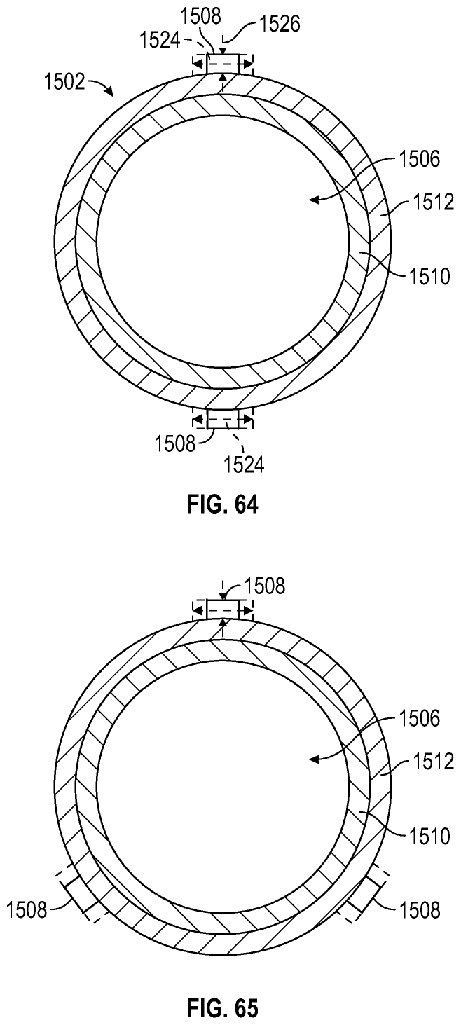

The elongate flat braid ribbon 1508 may have a length, and a width 1524 that extends circumferentially with respect to the elongate shaft 1502. The elongate flat braid ribbon 1508 may further have a radial thickness 1526 (marked in FIG. 64). In embodiments, the elongate flat braid ribbon 1508 may extend with its length parallel with the axis 1528 of the elongate shaft 1502. In other configurations, the elongate flat braid ribbon 1508 may have another orientation, such as extending in a spiral about the axis 1528 of the elongate shaft 1502 or another orientation.

One elongate flat braid ribbon 1508 may be utilized, or in embodiments a plurality of elongate flat braid ribbons may be utilized. One elongate flat braid ribbon 1508 may be positioned on the elongate shaft 1502 as desired. In embodiments in which a plurality of elongate flat braid ribbons are utilized, the ribbons may be positioned equally from each other, or at another configuration as desired. In an embodiment as shown in FIGS. 58 and 59, the ribbons may be positioned on opposite sides of the elongate shaft 1502. Such a configuration may be offset from a plane of flex of the elongate shaft 1502. In other embodiments, such as shown in FIG. 65, three ribbons may be equally spaced from each other to accommodate a variety of planes of flex of the elongate shaft 1502.

The elongate flat braid ribbon 1508 may be configured to enhance the tensile strength of the elongate shaft 1502, particularly during retraction of a portion of the elongate shaft 1502. For example, if the capsule 1500 is retracted and the elongate flat braid ribbon 1508 is positioned in the capsule 1500 then the tensile strength of the capsule 1500 may be enhanced. The elongate flat braid ribbon 1508 may be configured such that as a compressive force is applied to the elongate flat braid ribbon 1508, the width 1524 of the ribbon 1508 may increase without the thickness 1526 of the ribbon 1508 increasing. Such a feature may reduce the possibility of the diameter of the elongate flat braid ribbon 1508 increasing upon a compressive force being applied to the ribbon 1508. The width 1524 may increase circumferentially relative to the elongate shaft 1502 upon the elongate flat braid ribbon 1508 being axially compressed, and the radial thickness 1526 relative to the elongate shaft 1502 may not increase upon the elongate flat braid ribbon 1508 being axially compressed.

In embodiments, the elongate flat braid ribbon 1508 may be configured to have the ribbon's 1508 length extend upon a tensile force being applied to the ribbon 1508, yet reaching a stopping point at a certain length. Such a feature may strengthen the elongate shaft 1502 and reduce the possibility of tearing or other damage to the elongate shaft 1502 upon a tensile force being applied to the elongate shaft 1502.

In embodiments, the coil 1514 may extend over the elongate flat braid ribbon 1508 to secure the elongate flat braid ribbon 1508 to the elongate shaft 1502. FIG. 60, for example, illustrates the coil 1514 extending over the elongate flat braid ribbon 1508. The coil 1514 may further resist the radial expansion of the elongate flat braid ribbon 1508 upon a compressive force being applied to the elongate shaft 1502.

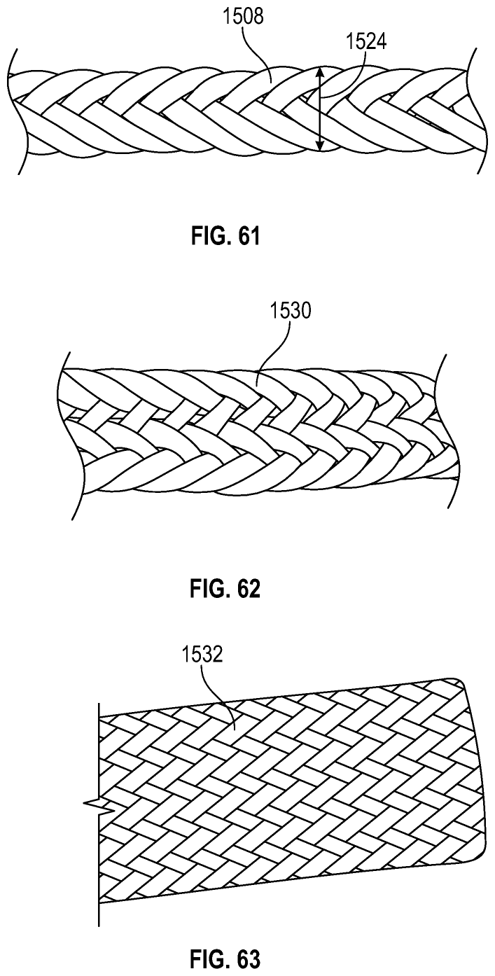

FIG. 61 illustrates a close up view of a configuration of the elongate flat braid ribbon 1508. The ribbon 1508 may include overlapping portions of cords that are configured to be compressed and have the width 1524 increase without an increase in the thickness 1526 (marked in FIG. 64) of the ribbon 1508. The cords may be configured to have a V-shape upon being braided together. Two or more cords may be utilized in embodiments in the braid (e.g., three cords, four cords, etc.). The cords in embodiments may comprise wires, cables, or other forms of cords in embodiments. In embodiments, the cords may comprise a metal or textile (e.g., cloth) material. In embodiments, a shape memory material such as nitinol may be utilized or other forms of shape memory materials.

FIG. 62 illustrates a close up view of a configuration of an elongate flat braid ribbon 1530 that may also be utilized according to embodiments herein. The elongate flat braid ribbon 1530 may include dual adjacent V-shaped braids that are braided together. Other configurations of flat braids may be utilized as desired. FIG. 63 for example illustrates a close up view of a configuration of an elongate flat braid ribbon 1532 including a plurality of adjacent V-shaped braids that are braided together. Other configurations of flat braids, aside from V-shaped braids may be utilized in embodiments as desired.

FIG. 64 illustrates a cross sectional view of the elongate shaft 1502 shown in FIG. 58, with two elongate flat braid ribbons 1508 positioned on opposite sides of the elongate shaft 1502. Features such as the coil 1514 and outer layers are excluded from view in FIG. 64 for clarity. The elongate flat braid ribbon 1508 may be configured to axially compress. The elongate flat braid ribbons 1508 may each be configured to have their width 1524 increase, without an increase in the radial thickness 1526 of the ribbons 1508 upon an axially compressive force being applied to the ribbons 1508. FIG. 65 illustrates a configuration in which three elongate flat braid ribbons 1508 may be equally spaced on the elongate shaft 1502. In embodiments, a plurality of elongate flat braid ribbons 1508 may extend along the elongate shaft 1502, and may include at least two elongate flat braid ribbons 1508, or at least three elongate flat braid ribbons, or another amount (e.g., at least four ribbons, at least five ribbons, etc.).

In a method, the elongate shaft 1502 of a delivery system may be delivered to an implantation site. The elongate shaft 1502 may include an implant retention area for retaining an implant for deployment to the implantation site, and the elongate shaft may include an elongate flat braid ribbon 1508 extending along the elongate shaft 1502.

The elongate flat braid ribbons and other features disclosed in regard to FIGS. 58-65 may be utilized with any embodiments of delivery system disclosed herein. The embodiments of elongate flat braid ribbons and other features disclosed in regard to FIGS. 58-65 may be utilized solely, or in combination with features of other systems, apparatuses, or methods disclosed herein.

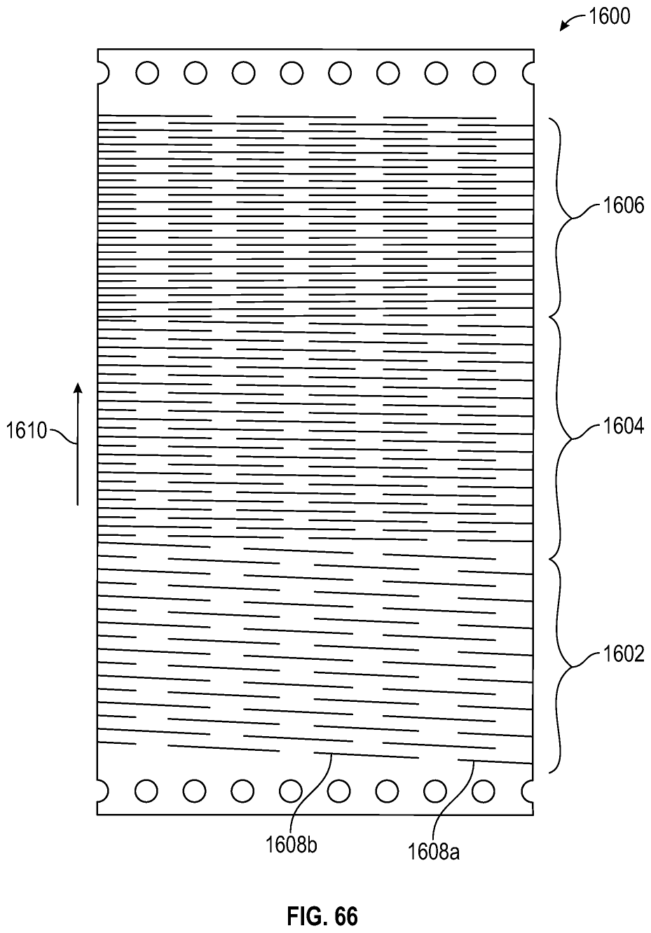

FIG. 66 illustrates an embodiment of a hypotube 1600 that may be utilized according to embodiments herein. The hypotube 1600 may be positioned within a capsule of a delivery system. The capsule may extend over an implant retention area for retaining an implant according to embodiments herein. The capsule may be part of an elongate shaft having the implant retention area for retaining an implant. The hypotube may be coupled to the elongate shaft and extend along the capsule. The hypotube 1600 is shown as a flat pattern in FIG. 66.

The hypotube 1600 may include a cut pattern. The hypotube 1600 may include at least three portions, including a proximal portion 1602, a middle portion 1604, and a distal portion 1606. The middle portion 1604 may be positioned between the proximal portion 1602 and the distal portion 1606. Each of the proximal portion 1602, the middle portion 1604, and the distal portion 1606 may include a spiral cut pattern. The spiral cut pattern may comprise an interrupted spiral cut pattern according to embodiments, in which the spiral cut is interrupted between circumferentially adjacent cuts (such cuts are marked as cuts 1608a and 1608b for example). Thus, material of the hypotube 1600 exists between the circumferentially adjacent cuts. Each of the proximal portion 1602, the middle portion 1604, and the distal portion 1606 may include an interrupted spiral cut pattern. In an axial dimension 1610 of the hypotube 1600, the spiral cut pattern of the proximal portion 1602, the middle portion 1604, and the distal portion 1606 each includes a repeating pattern of cuts circumferentially offset along the axial dimension 1610 of the hypotube 1600.

The pitch of the spiral cut pattern of the proximal portion 1602 (which may be referred to as a first portion) is different from the pitch of the spiral cut pattern of the middle portion 1604 (which may be referred to as a second portion), which is different from the pitch of the spiral cut pattern of the distal portion 1606 (which may be referred to as a third portion) and is different from the pitch of the proximal portion 1602. In embodiments, the pitch of the spiral cut pattern of the distal portion 1606 may be lesser than the pitch of the spiral cut pattern of the middle portion 1604, which may be lesser than the pitch of the spiral cut pattern of the proximal portion 1602. As such, the distance between the rows of cuts in the axial dimension 1610 may be greater in the proximal portion 1602 than in the middle portion 1604, which is greater than in the distal portion 1606. Each portion 1602, 1604, 1606 may have a different flexibility than the other portions.

The proximal portion 1602, in embodiments, may have a lesser flexibility than the middle portion 1604 and the distal portion 1606. The middle portion 1604, in embodiments, may have a lesser flexibility than the distal portion 1606. The varied flexibility may produce a desired flexibility profile for the hypotube 1600. For example, in an embodiment in which the hypotube 1600 comprises a portion of the capsule of a delivery system, the distal portion 1606 may correspond to a distal portion of the capsule, the middle portion 1604 may correspond to a middle portion of the capsule, and the proximal portion 1606 may correspond to a proximal portion of the capsule. It may be desirable for the distal portion of the capsule to have a greater flexibility than a middle or proximal portion of the capsule as the distal portion of the capsule may be required to flex at a greater ease than the middle or proximal portion. The proximal portion may be desired to be stiffer, and thus may have less flexibility than the middle or distal portions. For example, in an embodiment in which the capsule is bent, or extends over a bend in a rail assembly, the distal portion 1606 may be required to flex more easily than a proximal portion 1602, and thus may be more flexible than the proximal portion 1602.

In embodiments, distal portion 1606 may be configured to be retracted over a bend in the delivery system, which may comprise a bend in a rail assembly of the delivery system. For example, as disclosed herein, the elongate shaft may include a rail shaft that is configured to form at least one bend. The capsule and hypotube 1600 may be configured to be slid distally relative to the rail shaft. The distal portion may be configured to be retracted over the at least one bend.

In embodiments, the proximal portion 1602 may be adjacent to the middle portion 1604, which may be adjacent to the distal portion 1606 as shown in FIG. 66. The spiral cut pattern may be continuous from the proximal portion 1602 to the middle portion 1604 and then to the distal portion 1606. The spiral cut pattern of the proximal portion 1602 may be continuous with the spiral cut pattern of the middle portion 1604, and the spiral cut pattern of the middle portion 1604 may be continuous with the spiral cut pattern of the distal portion 1606. In embodiments, other configurations may be utilized.

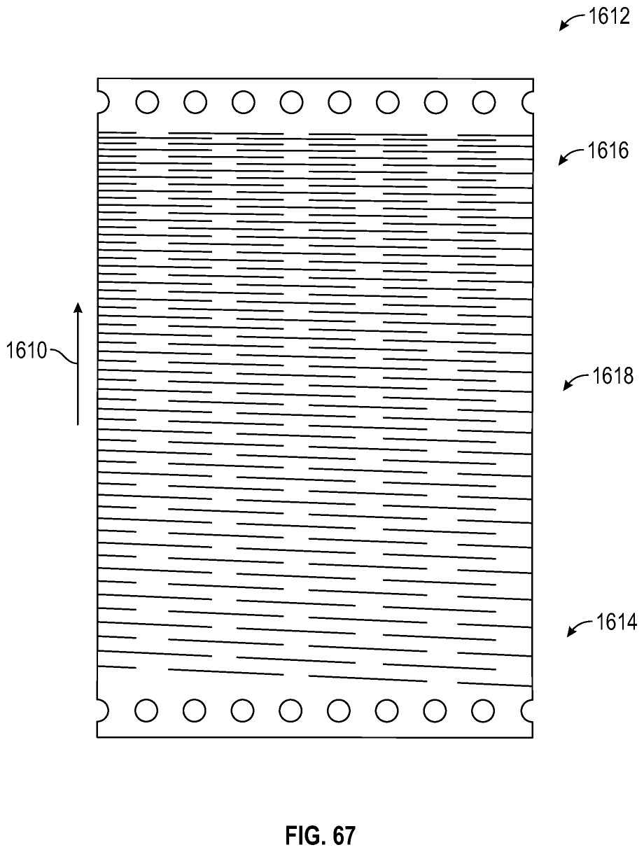

FIG. 67 illustrates an embodiment in which the hypotube 1612 has a spiral cut pattern having a continuous change in pitch from a proximal portion 1614 to a distal portion 1616. As such, the hypotube 1612 may include a middle portion 1618 incorporating this continuous change in pitch. The pitch at each portion may be different, with the proximal portion 1614 having a spiral cut pattern with a pitch, the middle portion 1618 having a spiral cut pattern with a pitch that is different from and less than the pitch of the proximal portion 1614, and the distal portion 1616 having a spiral cut pattern with a pitch that is different from and less than the pitch of the middle portion 1618 and the proximal portion 1614. The spiral cut pattern may extend continuously between the portions 1614, 1616, 1618. Each row along the axial dimension 1610 may have a different pitch.

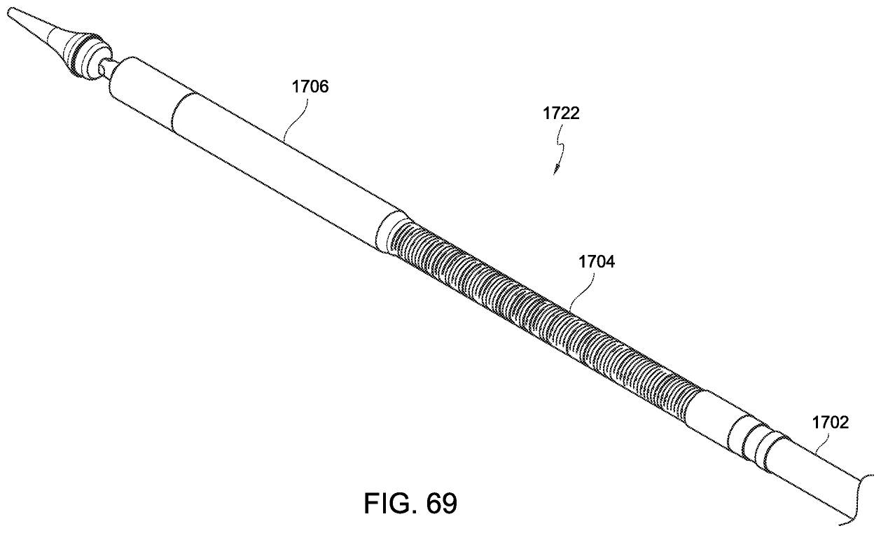

The hypotubes disclosed in FIGS. 66 and 67 may be utilized in any embodiment of delivery system disclosed herein. In embodiments, the hypotubes may comprise a portion of a capsule as disclosed herein. For example, in embodiments the hypotubes may comprise the hypotube 1512 disclosed in regard to FIG. 58. The hypotubes may further be utilized with the capsule 1706 shown in FIG. 69 for example.

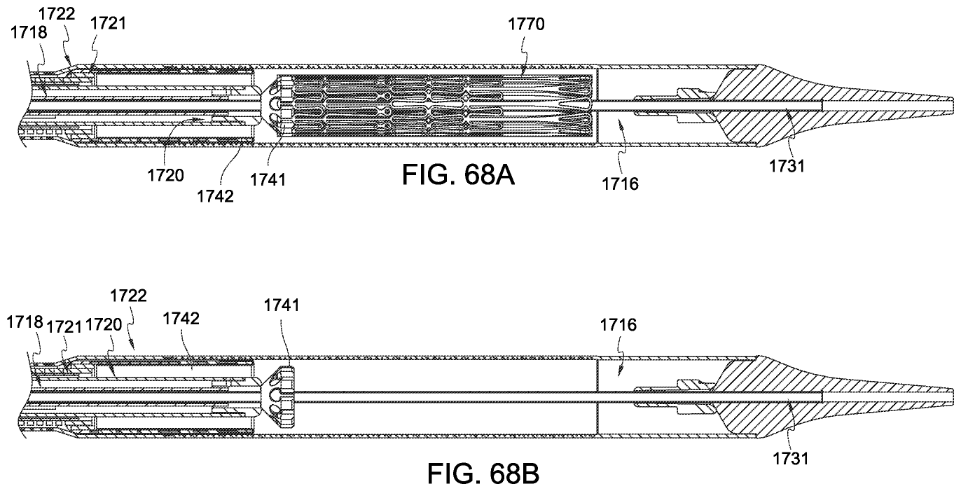

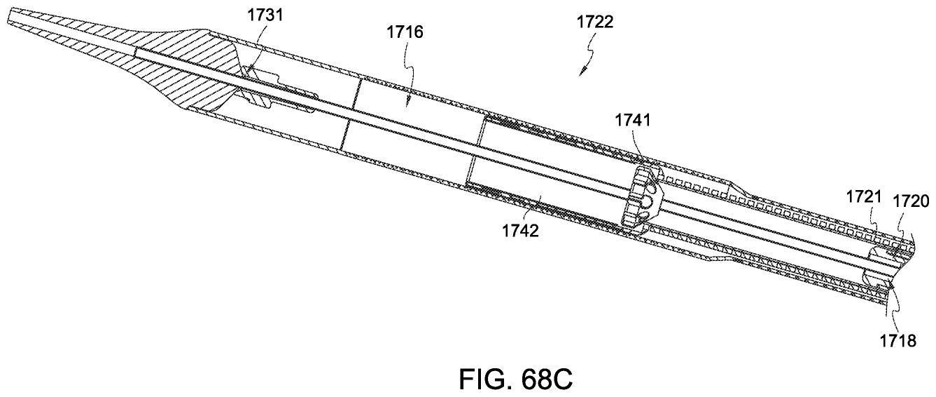

FIGS. 68A-75 illustrate features of a delivery system that may be utilized according to embodiments herein. The delivery system of FIGS. 68A-75 may incorporate any of the features disclosed in embodiments herein, including either of the hypotubes 1600, 1612 shown in FIGS. 66, 67. Features of delivery systems, methods, and other apparatuses are disclosed in U.S. patent application Ser. No. 16/028,172, filed on Jul. 5, 2018, and published as U.S. Patent Publication No. 2019/0008640, the entire contents of which are incorporated by reference herein.

FIG. 68A further shows an example of the implant 1770 that can be inserted into the delivery system, specifically into the implant retention area 1716. For ease of understanding, in FIG. 68A, the prosthesis is shown with only the bare metal frame illustrated. The prosthesis or implant 1770 can take any number of different forms.

Additional details and example designs for a prosthesis are described in U.S. Pat. Nos. 8,403,983, 8,414,644, 8,652,203 and U.S. Patent Publication Nos. 2011/0313515, 2012/0215303, 2014/0277390, 2014/0277422, 2014/0277427, 2018/0021129, and 2018/0055629, the entirety of these patents and publications are hereby incorporated by reference and made a part of this specification. Further details and embodiments of a replacement heart valve or prosthesis and its method of implantation are described in U.S. Publication Nos. 2015/0328000 and 2016/0317301 the entirety of each of which is hereby incorporated by reference and made a part of this specification.

The delivery system can be relatively flexible. In some embodiments, the delivery system is particularly suitable for delivering a replacement heart valve to a mitral valve location through a transseptal approach (e.g., between the right atrium and left atrium via a transseptal puncture). The delivery system, however, may be suitable for delivering a replacement heart valve to a tricuspid valve location, among other locations.

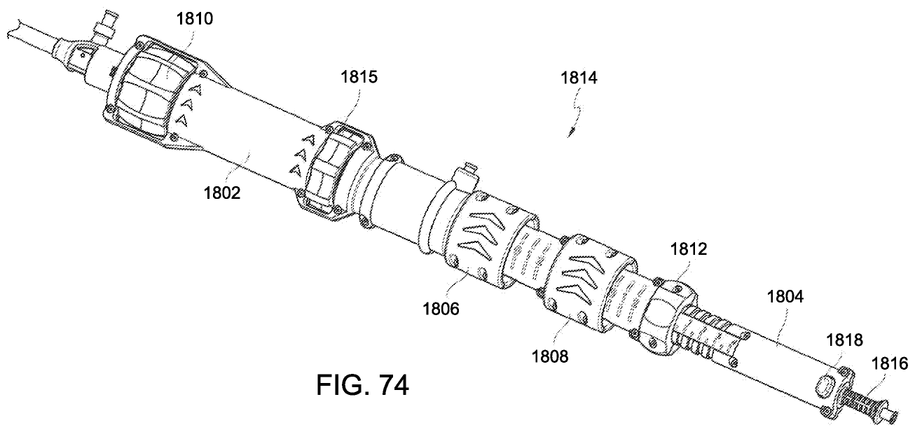

The delivery system can include a shaft assembly or elongate shaft comprising a proximal end and a distal end, wherein a handle 1814 (marked in FIG. 74) is coupled to the proximal end of the elongate shaft. The elongate shaft can be used to hold the implant 1770 for advancement of the same through the vasculature to a treatment location. The elongate shaft can include an implant retention area 1716 at its distal end that can be used for this purpose. In some embodiments, the elongate shaft can hold an expandable prosthesis in a compressed state at implant retention area 1716 for advancement of the implant 1770 within the body. The elongate shaft may then be used to allow controlled expansion of the implant 1770 at the treatment location. In some embodiments, the elongate shaft may be used to allow for sequential controlled expansion of the implant 1770 as discussed in detail below. The implant retention area 1716 is shown in FIGS. 68A-B at the distal end of the delivery system, but may also be at other locations. In some embodiments, the implant 1770 may be rotated in the implant retention area 1716, such as through the rotation of the inner shaft assembly 1718 discussed herein.

As shown in cross-sectional view of FIGS. 68A-68B, the distal end of the delivery system can include one or more subassemblies such as an outer sheath assembly 1722, a mid shaft assembly 1721, a rail assembly 1720, an inner shaft assembly 1718, and a nose cone assembly 1731 as will be described in more detail below. In some embodiments, the delivery system may not have all of the assemblies disclosed herein. For example, in some embodiments a full mid shaft assembly may not be incorporated into the delivery system. In some embodiments, the assemblies disclosed below may be in a different radial order than is discussed.

In particular, embodiments of the disclosed delivery system can utilize a steerable rail in the rail assembly 1720 for steering the distal end of the delivery system, allowing the implant to be properly located in a patient's body. As discussed in detail below, the steerable rail can be, for example, a rail shaft that extends through the delivery system from the handle 1814 (marked in FIG. 74) generally to the distal end. In some embodiments, the steerable rail has a distal end that ends proximal to the implant retention area 1716. A user can manipulate the bending of the distal end of the rail, thereby bending the rail in a particular direction. In preferred embodiments, the rail has more than one bend along its length, thereby providing multiple directions of bending. As the rail is bent, it presses against the other assemblies to bend them as well, and thus the other assemblies of the delivery system can be configured to steer along with the rail as a cooperating single unit, thus providing for full steerability of the distal end of the delivery system.

Once the rail is steered into a particular location in a patient's body, the implant 1770 can be advanced along or relative to the rail through the movement of the other sheaths/shafts relative to the rail and released into the body. For example, the rail can be bent into a desired position within the body, such as to direct the implant 1770 towards the native mitral valve. The other assemblies (e.g., the outer sheath assembly 1722, the mid shaft assembly 1721, the inner assembly 1718, and the nose cone assembly 1731) can passively follow the bends of the rail. Further, the other assemblies (e.g., the outer sheath assembly 1722, the mid shaft assembly 1721, the inner assembly 1718, and the nose cone assembly 1731) can be advanced together (e.g., relatively together, sequentially with one actuator, simultaneously, almost simultaneously, at the same time, closely at the same time) relative to the rail while maintaining the implant 1770 in the compressed position without releasing or expanding the implant 1770 (e.g., within the implant retention area 1716). The other assemblies (e.g., the outer sheath assembly 1722, the mid shaft assembly 1721, the inner assembly 1718, and the nose cone assembly 1731) can be advanced distally or proximally together relative to the rail. In some embodiments, only the outer sheath assembly 1722, mid shaft assembly 1721, and inner assembly 1718 are advanced together over the rail. Thus, the nose cone assembly 1731 may remain in the same position. The assemblies can be individually, sequentially, or simultaneously, translated relative to the inner assembly 1718 in order to release the implant 1770 from the implant retention area 1716.

FIG. 68C illustrates the sheath assemblies, specifically the outer sheath assembly 1722, the mid shaft assembly 1721, the inner shaft assembly 1718, and the nose cone assembly 1731 having translated distally together along the rail assembly 1720, further details on the assemblies are below. In some embodiments, the outer sheath assembly 1722, the mid shaft assembly 1721, the inner shaft assembly 1718, and the nose cone assembly 1731 translate together (e.g., relatively together, sequentially with one actuator, simultaneously, almost simultaneously, at the same time, closely at the same time). This distal translation can occur while the implant 1770 remains in a compressed configuration within the implant retention area 1716.

As shown in FIGS. 68A-68C and as further shown in FIGS. 69-73, starting with the outermost assembly, the delivery system can include an outer sheath assembly 1722 forming a radially outer covering, or sheath, to surround an implant retention area 1716 and prevent the implant from radially expanding. Specifically, the outer sheath assembly 1722 can prevent radial expansion of the distal end of the implant from radially expanding. Moving radially inward, the mid shaft assembly 1721 can be composed of a mid shaft hypotube 1743 with its distal end attached to an outer retention member or outer retention ring 1742 for radially retaining a portion of the prosthesis in a compacted configuration, such as a proximal end of the implant 1770. The mid shaft assembly 1721 can be located within a lumen of the outer sheath assembly 1722. Moving further inwards, the rail assembly 1720 can be configured for steerability, as mentioned above and further described below. The rail assembly 1720 can be located within a lumen of the mid shaft assembly 1721. Moving further inwards, the inner shaft assembly 1718 can be composed of an inner shaft with its distal end attached to inner retention member or inner retention ring 1741 (such as a PEEK ring) for axially retaining the prosthesis, for example the proximal end of the prosthesis. The inner shaft assembly 1718 can be located within a lumen of the rail assembly 1720. Further, the most radially-inward assembly is the nose cone assembly 1731 which includes the nose cone shaft 1727 having its distal end connected to the nose cone 1728. The nose cone 1728 can have a tapered tip. The nose cone assembly 1731 is preferably located within a lumen of the inner shaft assembly 1718. The nose cone assembly 1731 can include a lumen for a guide wire to pass therethrough.

The elongate shaft 1712, and more specifically the nose cone assembly 1731, inner assembly 1718, rail assembly 1720, mid shaft assembly 1721, and outer sheath assembly 1722, can be collectively configured to deliver an implant 1770 positioned within the implant retention area 1716 (shown in FIG. 68A) to a treatment location. One or more of the subassemblies can then be moved to allow the implant 1770 to be released at the treatment location. For example, one or more of the subassemblies may be movable with respect to one or more of the other subassemblies. The handle 1814 can include various actuation mechanisms that can be used to control the movement of the various subassemblies as will also be described in more detail below. In this way, the implant 1770 can be controllably loaded onto the delivery system and then later deployed within the body. Further, the handle 1814 can provide steering to the rail assembly 1720, providing for bending/flexing/steering of the distal end of the delivery system.

As will be discussed below, the inner retention member 1741, the outer retention ring 1742, and the outer sheath assembly 1722 can cooperate to hold the implant 1770 in a compacted configuration. The inner retention member 1741 is shown engaging struts 1772 at the proximal end of the implant 1770 in FIG. 68A. For example, slots located between radially extending teeth on the inner retention member 1741 can receive and engage the struts 1772 which may end in mushroom-shaped tabs on the proximal end of the implant 1770. The mid shaft assembly 1721 can be positioned over the inner retention member 1741 so that the first end of the implant 1770 is trapped between the inner retention member 1741 and the outer retention ring 1742, thereby securely attaching it to the delivery system between the mid shaft assembly 1721 and the inner retention member 1741. The outer sheath assembly 1722 can be positioned to cover the second end of the implant.

The outer retention member 1742 may be attached to a distal end of the mid shaft hypotube 1743 which can in turn be attached to a proximal tube 1744 at a proximal end, which in turn can be attached at a proximal end to the handle 1814. The outer retention member 1742 can provide further stability to the implant 1770 when in the compressed position. The outer retention member 1742 can be positioned over the inner retention member 1741 so that the proximal end of the implant 1770 is trapped therebetween, securely attaching it to the delivery system. The outer retention member 1742 can encircle a portion of the implant 1770, in particular the first end, thus preventing the implant 1770 from expanding. Further, the mid shaft assembly 1721 can be translated proximally with respect to the inner assembly 1718 into the outer sheath assembly 1722, thus exposing a first end of the implant 1770 held within the outer retention member 1742. In this way the outer retention member 1742 can be used to help secure an implant 1770 to or release it from the delivery system. The outer retention member 1742 can have a cylindrical or elongate tubular shape, and may be referred to as an outer retention ring, though the particular shape is not limiting.

As shown in FIG. 68A, the distal anchors can be located in a delivered configuration where the distal anchors point generally distally (as illustrated, axially away from the main body of the prosthesis frame and away from the handle of the delivery system). The distal anchors can be restrained in this delivered configuration by the outer sheath assembly 1722. Accordingly, when the outer sheath 1722 is withdrawn proximally, the distal anchors can flip positions (e.g., bend approximately 180 degrees) to a deployed configuration (e.g., pointing generally proximally). FIG. 68A also shows the proximal anchors extending distally in their delivered configuration within the outer sheath assembly 1722. In other embodiments, the distal anchors can be held to point generally proximally in the delivered configuration and compressed against the body of the prosthesis frame.

The delivery system may be provided to users with an implant 1770 preinstalled. In other embodiments, the implant 1770 can be loaded onto the delivery system shortly before use, such as by a physician or nurse.

FIGS. 69-73 illustrate further views of delivery system with different assemblies translated proximally and described in detail.

Starting with the outermost assembly shown in FIG. 69, the outer sheath assembly 1722 can include an outer proximal shaft 1702 directly attached to the handle 1814 at its proximal end and an outer hypotube 1704 attached at its distal end. A capsule 1706 can then be attached generally at the distal end of the outer hypotube 1704. In some embodiments, the capsule 1706 can be 28 French or less in size. These components of the outer sheath assembly 1722 can form a lumen for the other subassemblies to pass through.

A capsule 1706 can be located at a distal end of the outer proximal shaft 1702. The capsule 1706 can be a tube formed of a plastic or metal material. In some embodiments, the capsule 1706 is formed of ePTFE or PTFE. In some embodiments, this capsule 1706 is relatively thick to prevent tearing and to help maintain a self-expanding implant in a compacted configuration. In some embodiments the material of the capsule 1706 is the same material as the coating on the outer hypotube 1704. As shown, the capsule 1706 can have a diameter larger than the outer hypotube 1704, though in some embodiments the capsule 1706 may have a similar diameter as the hypotube 1704. In some embodiments, the capsule 1706 may include a larger diameter distal portion and a smaller diameter proximal portion. In some embodiments, there may be a step or a taper between the two portions. The capsule 1706 can be configured to retain the implant 1770 in the compressed position within the capsule 1706.

The outer sheath assembly 1722 is configured to be individually slidable with respect to the other assemblies. Further, the outer sheath assembly 1722 can slide distally and proximally relative to the rail assembly 1720 together with the mid shaft assembly 1721, inner assembly 1718, and nose cone assembly 1731.

In an embodiment in which the capsule includes the hypotubes 1600, 1612, the distal portions of the hypotubes 1600, 1612 may be positioned at reference number 1706 in FIG. 69. The middle portions of the hypotubes 1600, 1612 may be positioned at the transition of the outer diameter between the portions marked by reference nos. 1706 and 1704 in FIG. 69. The proximal portions of the hypotubes 1600, 1612 may be positioned at the portion marked by reference no. 1704 in FIG. 69.

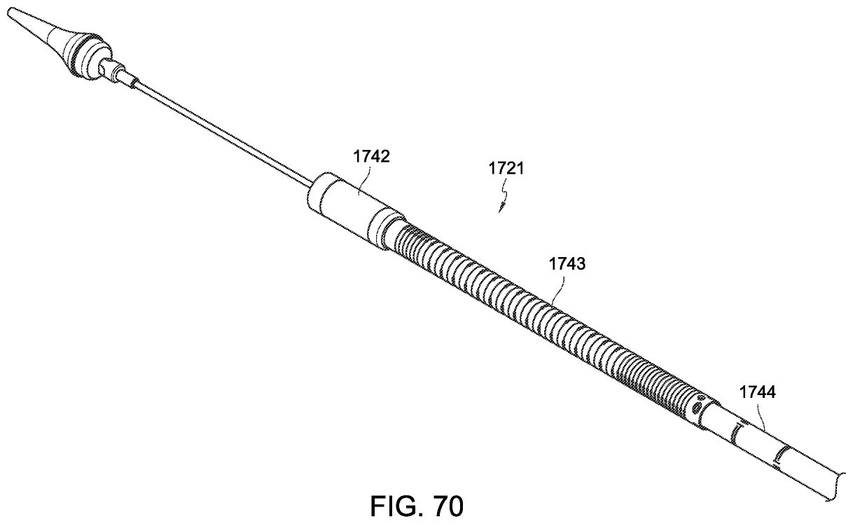

Moving radially inwardly, the next assembly is the mid shaft assembly 1721. FIG. 70 shows a similar view as FIG. 69, but with the outer sheath assembly 1722 removed, thereby exposing the mid shaft assembly 1721.

The mid shaft assembly 1721 can include a mid shaft hypotube 1743 generally attached at its proximal end to a mid shaft proximal tube 1744, which in turn can be attached at its proximal end to the handle 1814, and an outer retention ring 1742 located at the distal end of the mid shaft hypotube 1743. Thus, the outer retention ring 1742 can be attached generally at the distal end of the mid shaft hypotube 1743. These components of the mid shaft assembly 1721 can form a lumen for other subassemblies to pass through.

The outer retention ring 1742 can be configured as a prosthesis retention mechanism that can be used to engage with the implant 1770, as discussed with respect to FIG. 68A. For example, the outer retention ring 1742 may be a ring or covering that is configured to radially cover the struts on the implant 1770. The outer retention ring 1742 can also be considered to be part of the implant retention area 1716, and may be at the proximal end of the implant retention area 1716. With struts or other parts of an implant 1770 engaged with the inner retention member 1741, discussed below the outer retention ring 1742 can cover both the implant 1770 and the inner retention member 1741 to secure the implant 1770 on the delivery system. Thus, the implant 1770 can be sandwiched between the inner retention member 1741 of the inner shaft assembly 1718 and the outer retention ring 1742 of the mid shaft assembly 1721.

The mid shaft assembly 1721 is disposed so as to be individually slidable with respect to the other assemblies. Further, mid shaft assembly 1721 can slide distally and proximally relative to the rail assembly 1720 together with the outer sheath assembly 1722, the inner assembly 1718, and nose cone assembly 1731.

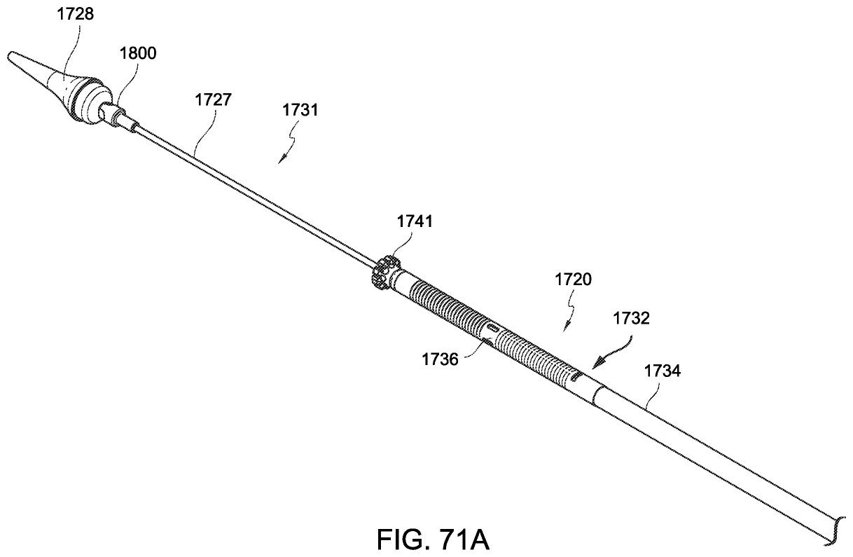

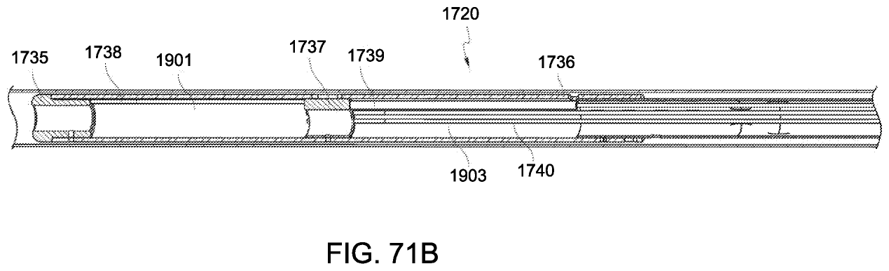

Next, radially inwardly of the mid shaft assembly 1721 is the rail assembly 1720. FIG. 71A shows approximately the same view as FIG. 70, but with the mid shaft assembly 1721 removed, thereby exposing the rail assembly 1720. FIG. 71B further shows a cross-section of the rail assembly 1720 to view the pull wires. The rail assembly 1720 can include a rail shaft 1732 (or rail) generally attached at its proximal end to the handle 1814. The rail shaft 1732 can be made up of a rail proximal shaft 1734 directly attached to the handle at a proximal end and a rail hypotube 1736 attached to the distal end of the rail proximal shaft 1734. The rail shaft 1732 may include a proximal rail shaft portion 1903 and a distal rail shaft portion 1901. The rail hypotube 1736 can further include an atraumatic rail tip at its distal end. Further, the distal end of the rail hypotube 1736 can abut a proximal end of the inner retention member 1741, as shown in FIG. 71A. In some embodiments, the distal end of the rail hypotube 1736 can be spaced away from the inner retention member 1741. These components of the rail shaft assembly 1720 can form a lumen for the other subassemblies to pass through.

As shown in FIG. 71B, attached to an inner surface of the rail hypotube 1736 are one or more pull wires which can be used apply forces to the rail hypotube 1736 and steer the rail assembly 1720. The pull wires can extend distally from the knobs in the handle 1814, discussed below, to the rail hypotube 1736. In some embodiments, pull wires can be attached at different longitudinal locations on the rail hypotube 1736, thus providing for multiple bending locations in the rail hypotube 1736, allowing for multidimensional steering.

In some embodiments, a distal pull wire 1738 can extend to a distal section of the rail hypotube 1736 and two proximal pull wires 1740 can extend to a proximal section of the rail hypotube 1736, however, other numbers of pull wires can be used, and the particular amount of pull wires is not limiting. For example, a two pull wires can extend to a distal location and a single pull wire can extend to a proximal location. In some embodiments, ring-like structures attached inside the rail hypotube 1736, known as pull wire connectors, can be used as attachment locations for the pull wires, such as proximal ring 1737 and distal ring 1735. In some embodiments, the rail assembly 1720 can include a distal pull wire connector 1735 and a proximal pull wire connector 1737. In some embodiments, the pull wires can directly connect to an inner surface of the rail hypotube 1736.

The distal pull wire 1738 can be connected (either on its own or through a connector 1735) generally at the distal end of the rail hypotube 1736. The proximal pull wires 1740 can connect (either on its own or through a connector 1737) at a location approximately one quarter, one third, or one half of the length up the rail hypotube 1736 from the proximal end. In some embodiments, the distal pull wire 1738 can pass through a small diameter pull wire lumen 1739 (e.g., tube, hypotube, cylinder) attached on the inside of the rail hypotube 1736. This can prevent the wires 1738 from pulling on the rail hypotube 1736 at a location proximal to the distal connection. Further, the lumen 1739 can act as compression coils to strengthen the proximal portion of the rail hypotube 1736 and prevent unwanted bending. Thus, in some embodiments the lumen 1739 is only located on the proximal half of the rail hypotube 1736. In some embodiments, multiple lumens 1739, such as spaced longitudinally apart or adjacent, can be used per distal wire 1738. In some embodiments, a single lumen 1739 is used per distal wire 1738. In some embodiments, the lumen 1739 can extend into the distal half of the rail hypotube 1736. In some embodiments, the lumen 1739 is attached on an outer surface of the rail hypotube 1736. In some embodiments, the lumen 1739 is not used.

For the pair of proximal pull wires 1740, the wires can be spaced approximately 180° from one another to allow for steering in both directions. Similarly, if a pair of distal pull wires 1738 is used, the wires can be spaced approximately 180° from one another to allow for steering in both directions. In some embodiments, the pair of distal pull wires 1738 and the pair of proximal pull wires 1740 can be spaced approximately 90° from each other. In some embodiments, the pair of distal pull wires 1738 and the pair of proximal pull wires 1740 can be spaced approximately 0° from each other. However, other locations for the pull wires can be used as well, and the particular location of the pull wires is not limiting. In some embodiments, the distal pull wire 1738 can pass through a lumen 1739 attached within the lumen of the rail hypotube 1736. This can prevent an axial force on the distal pull wire 1738 from creating a bend in a proximal section of the rail hypotube 1736.

The rail assembly 1720 is disposed so as to be slidable over the inner shaft assembly 1718 and the nose cone assembly 1731. In some embodiments, the outer sheath assembly 1722, the mid shaft assembly 1721, the inner shaft assembly 1718, and the nose cone assembly 1731 can be configured to slide together along or relative to the rail assembly 1720, such as proximally and distally with or without any bending of the rail assembly 1720. In some embodiments, the outer sheath assembly 1722, the mid shaft assembly 1721, the inner shaft assembly 1718, and the nose cone assembly 1731 can be configured to retain the implant 1770 in a compressed position when they are simultaneously slid along or relative to the rail assembly 1720.

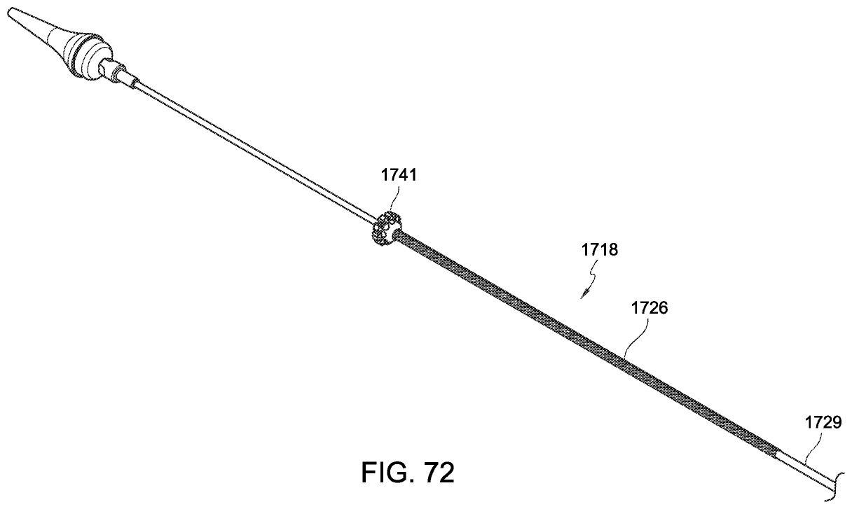

Moving radially inwards, the next assembly is the inner shaft assembly 1718. FIG. 72 shows approximately the same view as FIG. 71A, but with the rail assembly 1720 removed, thereby exposing the inner shaft assembly 1718.

The inner shaft assembly 1718 can include an inner shaft 1718 generally attached at its proximal end to the handle 1814, and an inner retention ring 1741 located at the distal end of the inner shaft 1718. The inner shaft 1718 itself can be made up of an inner proximal shaft 1729 directly attached to the handle 1814 at a proximal end and a distal section 1726 attached to the distal end of the inner proximal shaft 1729. Thus, the inner retention ring 1741 can be attached generally at the distal end of the distal section 1726. These components of the inner shaft assembly 1718 can form a lumen for the other subassemblies to pass through.

The inner retention member 1741 can be configured as a prosthesis retention mechanism that can be used to engage with the implant 1770, as discussed with respect to FIG. 68A. For example, the inner retention member 1741 may be a ring and can include a plurality of slots configured to engage with struts on the implant 1770. The inner retention member 1741 can also be considered to be part of the implant retention area 1716, and may be at the proximal end of the implant retention area 1716. With struts or other parts of an implant 1770 engaged with the inner retention member 1741, the outer retention ring 1742 can cover both the prosthesis and the inner retention member 1741 to secure the prosthesis on the delivery system. Thus, the implant 1770 can be sandwiched between the inner retention member 1741 of the inner shaft assembly 1718 and the outer retention ring 1742 of the mid shaft assembly 1721.

The inner shaft assembly 1718 is disposed so as to be individually slidable with respect to the other assemblies. Further, the inner assembly 1718 can slide distally and proximally relative to the rail assembly 1720 together with the outer sheath assembly 1722, mid shaft assembly 1721, and nose cone assembly 1731.

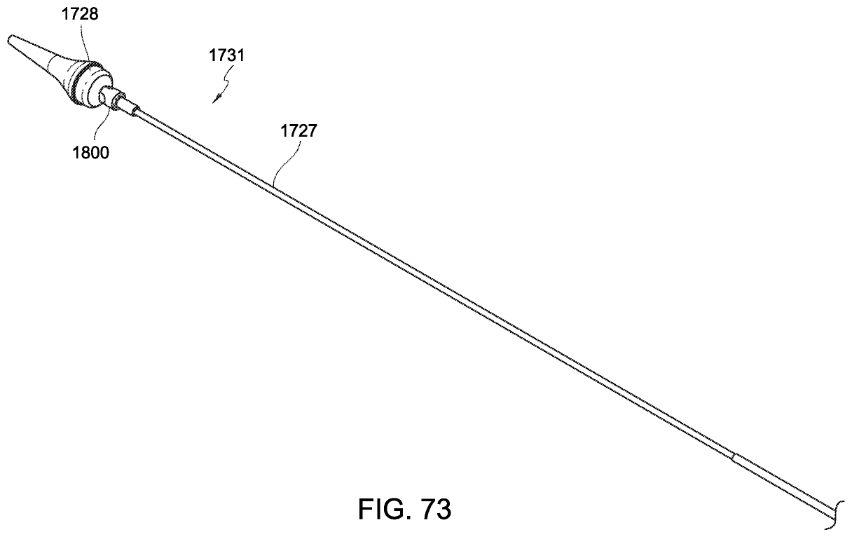

Moving further inwardly from the inner shaft assembly 1718 is the nose cone assembly 1731 also seen in FIG. 73. This may be a nose cone shaft 1727, and in some embodiments, may have a nose cone 1728 on its distal end. The nose cone 1728 can be made of polyurethane for atraumatic entry and to minimize injury to venous vasculature. The nose cone 1728 can also be radiopaque to provide for visibility under fluoroscopy.

The nose cone shaft 1727 may include a lumen sized and configured to slidably accommodate a guide wire so that the delivery system can be advanced over the guide wire through the vasculature. However, embodiments of the system discussed herein may not use a guide wire and thus the nose cone shaft 1727 can be solid. The nose cone shaft 1727 may be connected from the nose cone 1728 to the handle, or may be formed of different segments such as the other assemblies. Further, the nose cone shaft 1727 can be formed of different materials, such as plastic or metal, similar to those described in detail above.

In some embodiments, the nose cone shaft 1727 includes a guide wire shield 1800 located on a portion of the nose cone shaft 1727.

The nose cone assembly 1731 is disposed so as to be individually slidable with respect to the other assemblies. Further, the nose cone assembly 1731 can slide distally and proximally relative to the rail assembly 1720 together with the outer sheath assembly 1722, mid shaft assembly 1721, and inner assembly 1718.

In some embodiments, one or more spacer sleeves (not shown) can be used between different assemblies of the delivery system. For example, a spacer sleeve can be located concentrically between the mid shaft assembly and the rail assembly 1720, generally between the mid 1743 and rail hypotubes 1736. In some embodiments, the spacer sleeve can be generally embedded in the hypotube 1743 of the mid shaft assembly 1721, such as on an inner surface of the mid shaft assembly 1721. In some embodiments, a spacer sleeve can be located concentrically between the rail assembly 1720 and the inner assembly 1718, generally within the rail hypotube 1736. In some embodiments, a spacer sleeve can be used between the outer sheath assembly 1722 and the mid shaft assembly 1721. In some embodiments, a spacer sleeve can be used between the inner assembly 1718 and the nose cone assembly 1731. In some embodiments, 4, 3, 2, or 1 of the above-mentioned spacer sleeves can be used. The spacer sleeves can be used in any of the above positions.

As discussed above, the outer sheath assembly 1722, the mid shaft assembly 1721, the inner assembly 1718, and the rail assembly 1720 can contain an outer hypotube 1704, a mid shaft hypotube, a distal section 1726, and a rail hypotube 1736, respectively. Each of these hypotubes/sections/shafts can be laser cut to include a number of slots, thereby creating a bending pathway for the delivery system to follow.

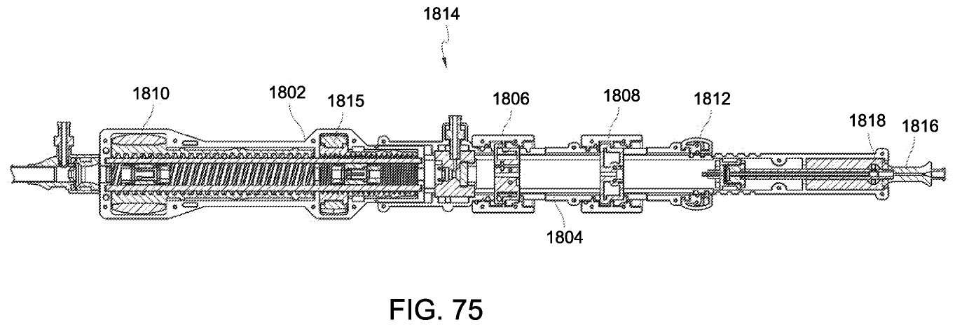

The handle 1814 is located at the proximal end of the delivery system. An embodiment of a handle 1814 is shown in FIG. 74. A cross-section of the handle 1814 is shown in FIG. 75. The handle 1814 can include a number of actuators, forming an actuation mechanism, such as rotatable knobs, that can manipulate different components of the delivery system. The operation of the handle 1814 is described with reference to delivery of a replacement valve prosthesis or implant 1770, though the handle 1814 and delivery system can be used to deliver other devices as well.

The handle 1814 is generally composed of two housings, a rail housing 1802 and a delivery housing 1804, the rail housing 1802 being circumferentially disposed around the delivery housing 1804. The inner surface of the rail housing 1802 can include a screwable section configured to mate with an outer surface of the delivery housing 1804. Thus, the delivery housing 1804 is configured to slide (e.g., screw) within the rail housing 1802, as detailed below. The rail housing 1802 generally surrounds about one half the length of the delivery housing 1804, and thus the delivery housing 1804 extends both proximally and distally outside of the rail housing 1802.

The rail housing 1802 can contain two rotatable knobs, a distal pull wire knob 1806 and a proximal pull wire knob 1808. However, the number of rotatable knobs on the rail housing 1802 can vary depending on the number of pull wires used. Rotation of the distal pull wire knob 1806 can provide a proximal force, thereby providing axial tension on the distal pull wires 1738 and causing the distal slotted section of the rail hypotube 1736 to bend. The distal pull wire knob 1806 can be rotated in either direction, allowing for bending in either direction, which can control anterior-posterior angles. Rotation of the proximal pull wire knob 1808 can provide a proximal force, and thus axial tension, on the proximal pull wires 1740, thereby causing the proximal slotted section 1733 of the rail hypotube 1736 to bend, which can control the medial-lateral angle. The proximal pull wire knob 1808 can be rotated in either direction, allowing for bending in either direction. Thus, when both knobs are actuated, there can be two bends in the rail hypotube 1736, thereby allowing for three-dimensional steering of the rail shaft 1732, and thus the distal end of the delivery system. Further, the proximal end of the rail shaft 1732 is connected on an internal surface of the rail housing 1802.

The bending of the rail shaft 1732 can be used to position the system, in particular the distal end, at the desired patient location, such as at the native tricuspid valve. In some embodiments, rotation of the pull wire knobs 1806/1808 can help steer the distal end of the delivery system to a desired position proximal a valve to be treated, for example a tricuspid or mitral valve.

Moving to the delivery housing 1804, the proximal ends of the inner shaft assembly 1718, outer sheath assembly 1722, mid shaft assembly 1721, and nose cone shaft assembly 1731 can be connected to an inner surface of the delivery housing 1804 of the handle 1814. Thus, they can move axially relative to the rail assembly 1720 and rail housing 1802.

A rotatable outer sheath knob 1810 can be located on the distal end of the delivery housing 1804, being distal to the rail housing 1802. Rotation of the outer sheath knob 1810 will pull the outer sheath assembly 1722 in an axial direction proximally, thus pulling the capsule 1706 away from the implant 1770 and releasing the distal end of implant 1770. Thus the outer sheath assembly 1722 is individually translated with respect to the other shafts in the delivery system. The distal end of the implant 1770 can be released first, while the proximal end of the implant 1770 can remain radially compressed between the inner retention member 1741 and the outer retention member 1742.

A rotatable mid shaft knob 1815 can be located on the delivery housing 1804, in some embodiments proximal to the rotatable outer sheath knob 1810, being distal to the rail housing 1802. Rotation of the mid shaft knob 1815 will pull the mid shaft assembly 1721 in an axial direction proximally, thus pulling the outer retention ring 1742 away from the implant 1770 and uncovering the inner retention member 1741 and the proximal end of the implant 1770, thereby releasing the implant 1770. Thus, the mid shaft assembly 1721 is individually translated with respect to the other shafts in the delivery system.

Located on the proximal end of the delivery housing 1804, and thus proximal to the rail housing 1802, can be a rotatable depth knob 1812. As the depth knob 1812 is rotated, the entirety of the delivery housing 1804 moves distally or proximally with respect to the rail housing 1802 which will remain in the same location. Thus, at the distal end of the delivery system, the inner shaft assembly 1718, outer sheath assembly 1722, mid shaft assembly 1721, and nose cone shaft assembly 1731 together (e.g., simultaneously) move proximally or distally with respect to the rail assembly 1720 while the implant 1770 remains in the compressed configuration. In some embodiments, actuation of the depth knob 1812 can sequentially move the inner shaft assembly 1718, outer sheath assembly 1722, mid shaft assembly 1721, and nose cone shaft assembly 1731 relative to the rail assembly 1720. In some embodiments, actuation of the depth knob 1812 can together move the inner shaft assembly 1718, outer sheath assembly 1722, and mid shaft assembly 1721 relative to the rail assembly 1720. Accordingly, the rail shaft 1732 can be aligned at a particular direction, and the other assemblies can move distally or proximally with respect to the rail shaft 1732 for final positioning while not releasing the implant 1770. The components can be advanced approximately 1, 2, 3, 5, 6, 7, 8, 9, or 10 cm along the rail shaft 1732. The components can be advanced more than approximately 1, 2, 3, 5, 6, 7, 8, 9, or 10 cm along the rail shaft 1732. An example of this is shown in FIG. 68C. The capsule 1706 and outer retention ring 1742 can then be individually withdrawn with respect to the inner assembly 1718 as discussed above, in some embodiments sequentially, releasing the implant 1770. The assemblies other than the rail assembly 1720 can then be withdrawn back over the rail shaft 1732 by rotating the depth knob 1812 in the opposite direction.

The handle 1814 can further include a mechanism (knob, button, handle) 1816 for moving the nose cone shaft 1727, and thus the nose cone 1728. For example, a knob 1816 can be a portion of the nose cone assembly 1731 that extends from a proximal end of the handle 1814. Thus, a user can pull or push on the knob 1816 to translate the nose cone shaft 1727 distally or proximally individually with respect to the other shafts. This can be advantageous for proximally translating the nose cone 1728 into the outer sheath assembly 1722/capsule 1706, thus facilitating withdraw of the delivery system from the patient.

In some embodiments, the handle 1814 can provide a lock 1818, such as a spring lock, for preventing translation of the nose cone shaft 1727 by the knob 1816 discussed above. In some embodiments, the lock 1818 can be always active, and thus the nose cone shaft 1727 will not move without a user disengaging the lock 1818. The lock can be, for example, a spring lock that is always engaged until a button 1818 on the handle 1814 is pressed, thereby releasing the spring lock and allowing the nose cone shaft 1727 to translate proximally/distally. In some embodiments, the spring lock 1818 allows one-way motion, either proximal or distal motion, of the nose cone shaft 1727 but prevents motion in the opposite direction.

The features of the delivery system disclosed in regard to FIGS. 68A-75 may be utilized with any embodiment of system, apparatus, or method disclosed herein.

As discussed, various forms of implants may be utilized with the embodiments disclosed herein, including prosthetic heart valves, or other forms of implants, such as stents or filters, or diagnostic devices, among others. The implants may be expandable implants configured to move from a compressed or undeployed state to an expanded or deployed state. The implants may be compressible implants configured to be compressed inward to have a reduced outer profile and to move the implant to the compressed or undeployed state. A crimping device as disclosed herein may assist in moving the implant to the compressed or undeployed state.

The delivery apparatuses as disclosed herein may be utilized for aortic, mitral, tricuspid, and pulmonary replacement and repair as well. The delivery apparatuses may comprise delivery apparatuses for delivery of other forms of implants, such as stents or filters, or diagnostic devices, among others.

The delivery apparatuses and the systems disclosed herein may be used in transcatheter aortic valve implantation (TAVI) or replacement of other native heart valves (e.g., mitral, tricuspid, or pulmonary). The delivery apparatuses and the systems disclosed herein may be utilized for transarterial access, including transfemoral access, to a patient's heart. The delivery apparatuses and systems may be utilized in transcatheter percutaneous procedures, including transarterial procedures, which may be transfemoral or transjugular. Transapical procedures, among others, may also be utilized.

Features of embodiments may be modified, substituted, excluded, or combined across embodiments as desired.

In addition, the methods herein are not limited to the methods specifically described, and may include methods of utilizing the systems and apparatuses disclosed herein. The steps of the methods may be modified, excluded, or added to, with systems, apparatuses, and methods disclosed herein.

The features of the embodiments disclosed herein may be implemented independently of the delivery apparatuses, or independent of other components disclosed herein. The various apparatuses of the system may be implemented independently.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of systems, apparatuses, and methods as disclosed herein, which is defined solely by the claims. Accordingly, the systems, apparatuses, and methods are not limited to that precisely as shown and described.

Certain embodiments of systems, apparatuses, and methods are described herein, including the best mode known to the inventors for carrying out the same. Of course, variations on these described embodiments will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described embodiments in all possible variations thereof is encompassed by the systems, apparatuses, and methods unless otherwise indicated herein or otherwise clearly contradicted by context.

Groupings of alternative embodiments, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A crimping device for an implant, the crimping device comprising:
a body having an inner surface forming a funnel extending from a wide end portion to a narrow end portion and surrounding a cavity for receiving the implant, the body including an opening at the narrow end portion and including a plurality of bumps on the inner surface that are spaced from each other to form a plurality of guide channels on the inner surface, each of the plurality of guide channels configured for a portion of the implant to slide in to guide the implant at the opening.

2. The crimping device of claim 1, wherein the plurality of guide channels are positioned proximate the opening.

3. The crimping device of claim 1, wherein the plurality of guide channels surround the opening.

4. The crimping device of claim 1, wherein the plurality of guide channels extend radially outward from the opening.

5. The crimping device of claim 1, wherein the plurality of guide channels extend at an angle relative to a plane of the opening.

6. The crimping device of claim 1, wherein the plurality of guide channels extend longitudinally in a direction from the narrow end portion to the wide end portion.

7. The crimping device of claim 1, wherein the body includes an actuator and a pressing body, the actuator configured to move the pressing body to press the implant in a direction towards the opening.

8. The crimping device of claim 1, wherein the plurality of guide channels are each configured for struts of the implant to slide in to guide the struts at the opening.

9. The crimping device of claim 8, wherein the plurality of guide channels are each configured to space the struts equally at the opening.

10. The crimping device of claim 9, wherein the struts each include an end tab portion.

11. A system comprising:
a prosthetic heart valve including prosthetic valve leaflets and a frame including end couplers for coupling with a delivery apparatus for the prosthetic heart valve; and
a crimping device configured to crimp the prosthetic heart valve, the crimping device including a body having an inner surface forming a funnel extending from a wide end portion to a narrow end portion and surrounding a cavity for receiving the prosthetic heart valve, the body including an opening at the narrow end portion and including a plurality of guide channels positioned proximate the opening and each extending radially outward from the opening, the plurality of guide channels configured to space the end couplers from each other at the opening to couple to the delivery apparatus.

12. The system of claim 11, wherein the end couplers comprise end tab portions on struts of the frame, the end tab portions configured to engage receiving portions of a coupler of the delivery apparatus.

13. The system of claim 11, wherein the plurality of guide channels are configured to space the end couplers equally from each other at the opening.

14. The system of claim 11, further comprising a pressing body configured to press the prosthetic heart valve distally towards the opening and including flanges, and wherein the body surrounds a longitudinal axis and includes slots extending longitudinally along the longitudinal axis for the flanges of the pressing body to slide within.

15. The system of claim 11, wherein a plurality of protrusions on the inner surface that are spaced from each other form the plurality of guide channels on the inner surface.

16. A crimping device for an implant, the crimping device comprising:
a body having an inner surface forming a funnel extending from a wide end portion to a narrow end portion and surrounding a cavity for receiving the implant, the body including an opening at the narrow end portion and including a plurality of guide channels positioned proximate the opening and each extending radially outward from the opening, the plurality of guide channels configured to space end couplers of the implant from each other at the opening for coupling to a delivery apparatus for the implant.

17. The crimping device of claim 16, wherein the plurality of guide channels are configured to space the end couplers of the implant equally from each other at the opening.

18. The crimping device of claim 16, wherein the body surrounds a longitudinal axis and includes a proximal portion and a distal portion, the inner surface at the distal portion having a greater surface angle relative to the longitudinal axis than the inner surface at the proximal portion, and the plurality of guide channels are positioned on the inner surface at the distal portion.

19. The crimping device of claim 16, wherein the plurality of guide channels are spaced equally from each other.

20. The crimping device of claim 16, wherein a plurality of protrusions on the inner surface that are spaced from each other form the plurality of guide channels on the inner surface.
